(19) United States
(12) Patent Application Publication
(10) Pub. No.: US 2004/0152769 A1
(43) Pub. Date: Aug. 5, 2004

(54) Modified carbamate-containing prodrugs and methods of synthesizing same

(76) Inventors: Nnochiri Nkem Ekwuribe, Cary, NC (US); Jennifer Riggs-Sauthier, Raleigh, NC (US); Tatyana A. Dyakonov, Durham, NC (US)

Correspondence Address: MYERS BIGEL SIBLEY & SAJOVEC, PO BOX 37428, RALEIGH, NC, 27627, US

(21) Appl. No.: 10/703,647

(22) Filed: Nov. 7, 2003

Related U.S. Application Data
(60) Provisional application No. 60/424,796, filed on Nov. 9, 2002.
(60) Provisional application No. 60/483,676, filed on Jun. 30, 2003.

(51) Int. Cl. A61K 31/66; A61K 31/325; A61K 31/16

(52) U.S. Cl. 514/478; 514/615; 514/114

(57) ABSTRACT

Prodrugs having a hydrolyzable carbamate moiety, compositions including the prodrugs, methods of preparing the prodrugs and methods of treatment using the prodrugs are disclosed. The prodrug has the formula DC(X)XR, where D is a biologically active agent, X is O, S or NR′, and R is a moiety that modifies various properties of the biologically active agent. The biologically active agent either includes a functional group such as an amide, thioamide, imide, thioimide, urea, thiourea, carbamate, thiocarbamate, sulfonamide, or sulfonimide group, or includes a hydroxy, amine, carboxylic acid or thiol group that is modified to include such a group. An NH group from the biologically active agent can be coupled to an activated form the C(X)XR moiety to form the prodrugs described herein. Relative to a conventional carbamate group, the presence of the additional carbonyl or sulfonyl group makes the carbamate group more susceptible to hydrolysis. The prodrugs are more stable in certain environments than the biologically active agent, and can permit the drugs to be administered orally, in those embodiments where the biologically active agent must otherwise be administered by injection or intraveneous administration.

### RELATED APPLICATION DATA

This application claims the benefit of U.S. Provisional Application Serial No. 60/424,796, filed Nov. 9, 2002, and U.S. Provisional Application Serial No. 60/483,676, filed Jun. 30, 2003, the disclosures of which are incorporated herein by reference in their entireties.

### 1 FIELD OF THE INVENTION

The present invention relates to carbamate prodrugs, methods of synthesizing such carbamate prodrugs, and methods of treatment employing the use of such carbamate prodrugs.

### 2 BACKGROUND OF THE INVENTION

Prodrug design represents an approach to drug delivery often employed to mask undesirable drug properties including, but not limited to, low bioavailability, lack of site specificity, chemical instability, toxicity, immunogenecity, and factors contributing to poor patient compliance such as bad taste, odor, or undesirable modes of administration. Generally, prodrugs are chemical derivatives that can be metabolized in vivo to provide active drug molecules capable of exerting a therapeutic effect.

Current prodrugs, however, generally have shortcomings that limit their practical applications such as the requirement for specific enzyme digestion, which may make the prodrug unsuitable or at least less useful for in vivo use; the generation of toxic intermediates; and complex and/or costly synthesis routes. It is desirable to provide new prodrugs with improved characteristics relative to prodrugs in the prior art, as well as methods for making and using such prodrugs. The present invention provides such prodrugs and methods.

### 3 SUMMARY OF THE INVENTION

Prodrug compounds comprising a modified carbamate linkage are disclosed. The carbamate linkage has been modified from a normal carbamate linkage (—O—C(O)—NH—) such that the H in an N—H bond is replaced with a link to another functional group, such as an amide, thioamide, imide, thioimide, urea, thiourea, carbamate, thiocarbamate and the like (i.e., the nitrogen in the NH bond from the carbamate is also a nitrogen in the amide, thioamide, imide, thioimide, urea, thiourea, carbarnate, thiocarbamate and the like). The O in the carbonyl in the carbamate moiety can be replaced with an NH, N-alkyl, N-aryl, or S. The resulting moieties can have improved biodegradability, and accordingly, provide improved drug release characteristics, e.g., relative to prodrugs including a standard carbamate moiety.

In contrast to extant carbamate groups, which only allow one to form prodrugs of hydroxyl and/or amine containing compounds, the present technology permits one to functionalize compounds including carboxylic acid, amine, hydroxyl and/or thio groups, as well as amides, thioamides, imides, thioimides, ureas, thioureas, carbamates, thiocarbamates, sulfonamides, sulfonimides, phosphoramides and the like.

The prodrugs can be formed from any drug including these moieties, with the proviso that where a functional group exists, and it is not desired to form a carbamate-like moiety at that functional group, it may be desirable to protect the functional group, and possibly deprotect that functional group after the prodrug synthesis is complete.

The technology allows one to attach hydrophilic, lipophilic and/or amphiphilic polymers to the prodrug, which can aid in allowing the compounds to pass through the stomach without degradation, and/or protect labile peptides and proteins from enzymatic degradation. Particularly where a polyethylene glycol moiety is attached, the presence of two or more PEG units can provide increased protection from enzymes such as proteases that would otherwise adversely affect the protein or peptide.

The prodrugs can be used to treat any condition for which the parent compound possesses utility, and often permit oral administration of drugs that are otherwise only able to be administered by injection or by intravenous administration. In certain embodiments, the prodrugs can possess improved pharmaceutical characteristics, such as greater solubility, greater chemical stability, and/or higher bioavailability than the native drug. Further, in certain embodiments, the prodrugs can be prepared more easily and/or more cost effectively than other known prodrugs.

In one embodiment, the prodrugs have the following formula:

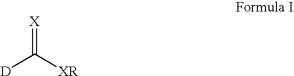

wherein:

X is O, S, or NR′,

R′ is, individually, hydrogen, alkyl, substituted alkyl, aryl, substituted aryl, arylalkyl, substituted arylalkyl, alkylaryl, or substituted alkylaryl,

“substituted,” as applied to alkyl, aryl, alkylaryl, and arylalkyl, refers to substituents selected from alkyl, alkenyl, heterocyclyl, cycloalkyl, aryl (including heteroaryl), alkylaryl, arylalkyl, halo (e.g., F, Cl, Br, or I), alkoxy, amine, trifluoroalkyl, such as trifluoromethyl, —CN, —NO₂, —SR′, —N₃, —C(═O)NR′₂, —NR′C(═O)R″, —C(═O)R′, —C(═O)OR′, —OC(═O)R′, —NR′SO₂R′, —OC(═O)NR′₂, —NR′C(═O)OR′, —SO₂R′, and —SO₂NR′₂,

D is a biologically active agent (“drug”) that includes a functional group, such as a urea, thiourea, amide, thioamide, imide, thioimide, carbamate, thiocarbamate, sulfonamide, sulfonimide, phosphoramide, and the like, or which originally included a hydroxyl, amine, thiol, and/or carboxylic acid group, where such group has been modified to be in the form of a urea, thiourea, amide, thioamide, imide, thioimide, carbamate, thiocarbamate, sulfonamide, sulfonimide, phosphoramide, and the like,

the linkage between D and the C═X moiety (such as a carbonyl) is through an —N— linkage, formed from an NH group present in the urea, thiourea, amide, thioamide, imide, thioimide, carbamate, thiocarbamate, sulfonamide, sulfonimide, phosphoramide, and the like before being coupled to the C═X moiety in Formula I, resulting in functional groups that are more readily hydrolyzable than traditional carbamate functional groups,

and R is a “modifying moiety.”

In a preferred embodiment, the —C(X)XR portion of Formula I is —C(O)OR, and the carbonyl moiety of the —C(O)OR group is coupled to an —NH— functionality of the urea, thiourea, amide, thioamide, imide, thioimide, carbamate, thiocarbamate, sulfonamide, sulfonimide, phosphoramide, moiety of D such that the amine functionality of D and the carbonyl moiety and the oxygen of the —C(O)OR group form a hydrolyzable carbamate moiety.

“Modifying moieties” modify various characteristics of the native drug in a manner that provides the prodrug with desired properties. For example, the moiety can modify the drug by providing the drug with improved stability in certain environments, increasing the drug's hydrophilicity or hydrophobicity, increasing the drug's ability to cross the cell membrane, increasing the drug's ability to cross the blood-brain barrier, or targeting the drug to a certain receptor, cell (for example, a tumor cell), tissue, or organ. In one embodiment, R is a moiety that affects the chemical stability of D, such that the prodrug is more chemically stable in a particular environment than the drug itself.

In some embodiments, such as imides, thioimides, sulfonimides, and the like, where there are two —C(O)—N—, —C(S)—N—, —C(NR′)—N—, and/or —SO₂—N— moieties, one of the linkages to the moieties can be to a drug, and another can be to a modifying moiety, R, as described herein.

A subset of the compounds of Formula 1 has the following formula:

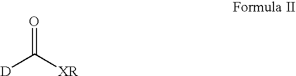

where D, X and R are as defined above.

Another subset of the compounds of Formula 1 has the following formula:

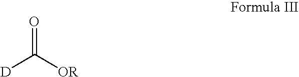

where D, X and R are as defined above.

The products have a different level of chemical stability and/or biological activity than the drugs themselves. In some environments, the prodrugs are more stable than the drugs themselves, and in such environments, one can achieve greater drug bioavailability. In other environments, the prodrugs are less stable than the native drugs, or may have the same stability as the native drugs, but the prodrug is inactive and/or exhibits reduced side effects relative to the drug itself. This can provide for sustained release of active drug without concomitant side effects associated with larger doses.

Pharmaceutical compositions including the prodrugs, methods of synthesizing the prodrugs, and methods of treatment using the prodrugs are also described.

### 4 BRIEF DESCRIPTION OF THE DRAWINGS

FIG. 1 illustrates a bar graph denoting the chemical hydrolysis of a prodrug according to embodiments of the present invention compared to the chemical hydrolysis of the parent compound after 0, 1.5, 3.0, 4.5, and 6.0 hours post-dose at pH 2, 7.4, and 8;

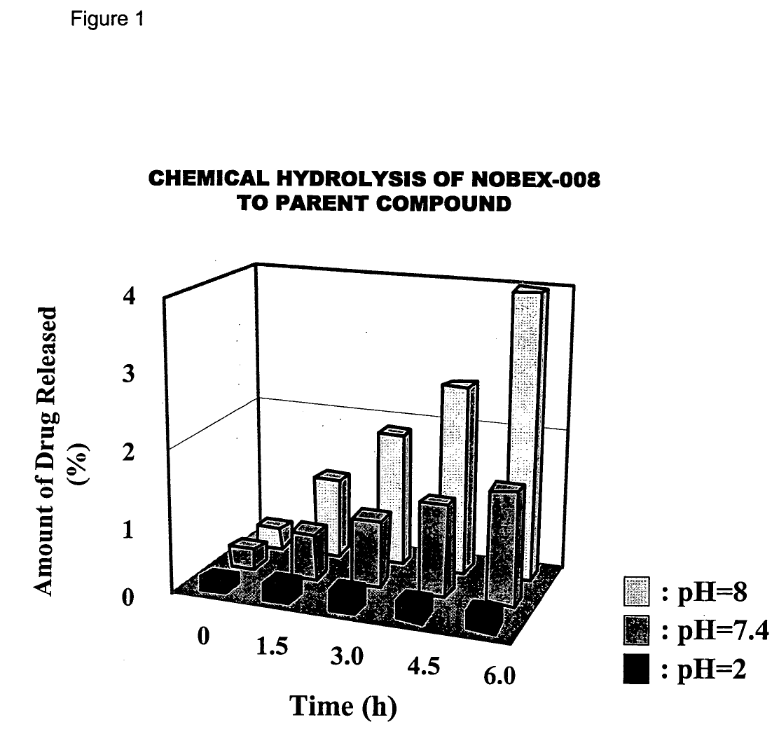

FIG. 2 illustrates a graph denoting the level of parent drug in rats dosed orally with prodrugs according to embodiments of the present invention analyzed by LC/MS/MS detection;

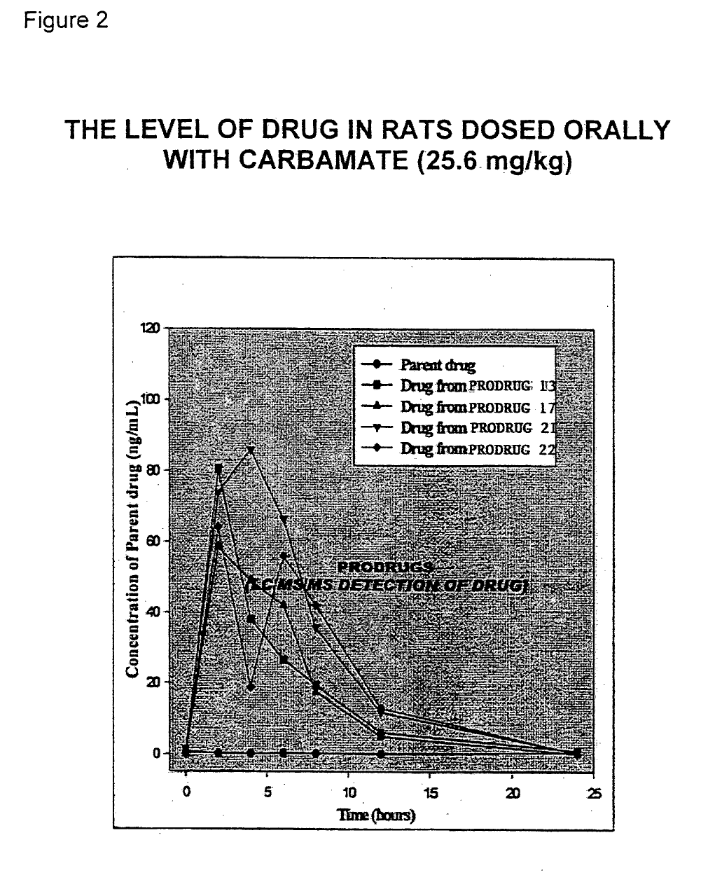

FIG. 3 illustrates a graph denoting the level of prodrug according to embodiments of the present invention in rat plasma after oral dosing at 0, 2, 4, 6, 8, and 10 hours post-dose compared to the level of parent drug in rat plasma after oral dosing at 0, 2, 4, 6, 8, and 10 hours post-dose; and

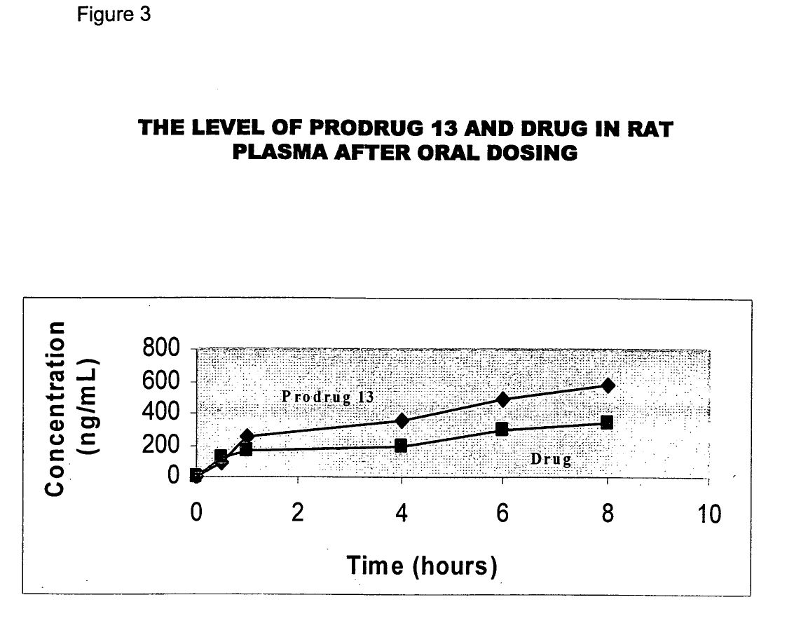

FIG. 4 illustrates a bar graph denoting bioavailability (AUC) of prodrugs according to embodiments of the present invention as a function of polyethylene glycol (PEG) chain length.

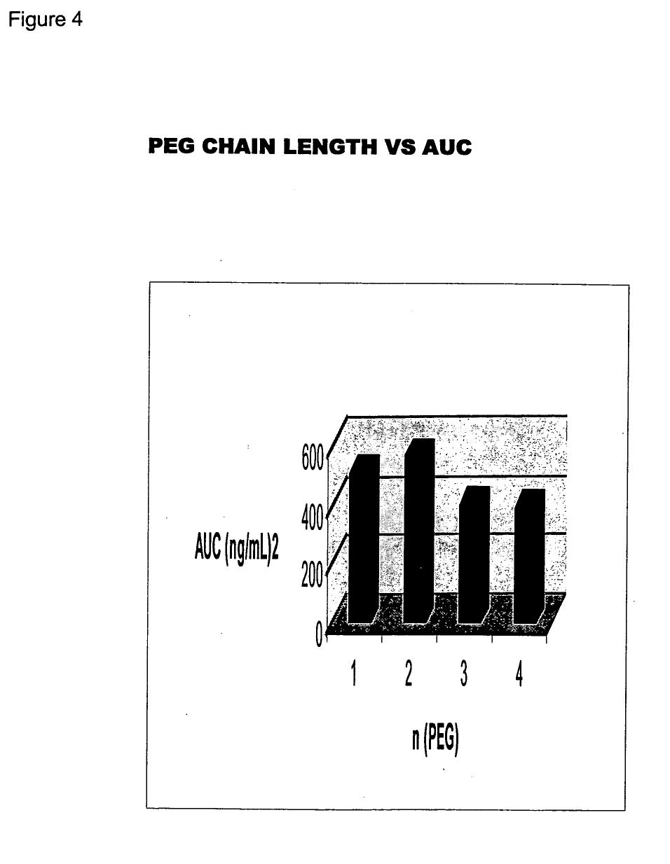

FIG. 5 is a schematic illustration of amine-containing drugs, various modifications of the amine-containing drugs, and the prodrugs formed from the modified amine-containing drugs.

### 5 DETAILED DESCRIPTION OF EMBODIMENTS

The invention will now be described with respect to embodiments described herein. It should be appreciated that the invention may be embodied in different forms and should not be construed as limited to the embodiments set forth herein. Rather, these embodiments are provided so that this disclosure will be thorough and complete, and will fully convey the scope of the invention to those skilled in the art.

5.1 Terminology and Definitions

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, and other references mentioned herein are incorporated by reference in their entirety, as are the package inserts of any branded drugs referred to herein by their brand names.

Singular forms “a”, “an”, and “the” are intended to include the plural forms as well, unless the context clearly indicates otherwise.

As used in the specification and the claims set forth herein, the following terms have the meanings indicated:

“Alkylaryl” refers to an alkyl moiety including an aryl substituent.

“Amphiphilic” means the ability to dissolve in both water and lipids, and the terms “amphiphilic moiety” and “amphipile” mean a moiety which is amphiphilic and/or which, when attached to a polypeptide or non-polypeptide drug, increases the amphiphilicity of the resulting conjugate, e.g., PEG-fatty acid oligomer, sugar-fatty acid oligomer.

“Aryl” or “aromatic” refer to 3 to 10, preferably 5 and 6-membered ring aromatic and heteroaromatic rings.

“Arylalkyl” refers to an aryl moiety including an alkyl substituent.

“Biologically active agent” refers to any therapeutic or pharmacologic agent that is conjugatable in the manner of the present invention (i.e., agents comprising an amide, thioamide, imide, thiomide, urea, thiourea, carbamate, thiocarbamate, sulfonamide, sulfonimide, phosphoramide, or similar functional group, or which include a hydroxyl, thiol, amine and/or carboxylic acid functional groups, where the hydroxyl, thiol, amine and/or carboxylic acid functional groups is modified to be in the form of one of the above functional groups). The terms “parent compound,” “native drug,” “unconjugated drug,” and “parent drug” are also used herein in contradistinction to the term “prodrug” and can refer to the “biologically active agent” as defined herein unless specifically indicated otherwise.

“C₁₋₆ alkoxy radicals” contain from 1 to 6 carbon atoms in a straight or branched chain, and also include C₃₋₆ cycloalkyl and alkoxy radicals that contain C₃₋₆ cycloalkyl moieties.

“Chemical stability” refers to the stability of a given compound in physiological environments. For example, chemical stability refers to the stability of the biologically active agent or prodrug in environments characterized by conditions such as, but not limited to, the presence of plasma, the presence of proteases, the presence of liver homogenate, the presence of acidic conditions, and the presence of basic conditions.

“Cycloalkyl radicals” contain from 3 to 8 carbon atoms. Examples of suitable cycloalkyl radicals include, but are not limited to, cyclopropyl, cyclobutyl, cyclopentyl, cyclohexyl, cycloheptyl and cyclooctyl.

“Effective amount” of a prodrug as provided herein refers to a nontoxic but sufficient amount of the prodrug to provide the desired therapeutic effect. As will be pointed out below, the exact amount required will vary from subject to subject, depending on age, general condition of the subject, the severity of the condition being treated, the particular biologically active agent administered, and the like. An appropriate “effective” amount in any individual case may be determined by one of ordinary skill in the art by reference to the pertinent texts and literature and/or by using routine experimentation.

“Halogen” is chlorine, iodine, fluorine or bromine.

“Heteroaryl radicals” contain from 3 to 10 members, preferably 5 or 6 members, including one or more heteroatoms selected from oxygen, sulfur and nitrogen. Examples of suitable 5-membered ring heteroaryl moieties include furyl, thienyl, pyrrolyl, imidazolyl, oxazolyl, thiazolyl, isoxazolyl, isothiazolyl, tetrazolyl, triazolyl and pyrazolyl. Examples of suitable 6-membered ring heteroaryl moieties include pyridinyl, pyrimidinyl, pyrazinyl and pyridazinyl, of which pyridinyl and pyrimidinyl are preferred.

“Heterocyclyl radicals” contain from 3 to 10 members including one or more heteroatoms selected from oxygen, sulfur and nitrogen. Examples of suitable heterocyclyl moieties include, but are not limited to, piperidinyl, morpholinyl, pyrrolidinyl, imidazolidinyl, pyrazolidinyl, isothiazolidinyl, thiazolidinyl, isoxazolidinyl, oxazolidinyl, piperazinyl, oxanyl (tetrahydropyranyl) and oxolanyl (tetrahydrofuranyl).

“Hydrolyzable” refers to bonds which are hydrolyzed under physiological conditions.

“Hydrophilic” means the ability to dissolve in water, and the term “hydrophilic moiety” or “hydrophile” refers to a moiety which is hydrophilic and/or which when attached to another chemical entity, increases the hydrophilicity of such chemical entity. Examples include, but are not limited to, sugars and polyalkylene moieties such as polyethylene glycol.

“Hydroxyl-containing drug,” “thiol-containing drug,” “amine-containing drug,” and “carboxyl-containing drug” refer to drugs that include hydroxyl, amine, thiol and carboxylic acid groups, respectively. It is to be understood that numerous drugs include more than one type of functional group, and can be properly characterized as more than one of the above.

“Lipophilic” means having an affinity for fat, such as chemicals that accumulate in fat and fatty tissues. “Lipophilic” further refers to the ability to dissolve in lipids and/or the ability to penetrate, interact with and/or traverse biological membranes, and the term, “lipophilic moiety” or “lipophile” means a moiety which is lipophilic and/or which, when attached to another chemical entity, increases the lipophilicity of such chemical entity.

“Lower alkyl” refers to substituted or unsubstituted alkyl moieties having from 1 to 6 carbon atoms.

“Pharmaceutically acceptable” with respect to a component, such as a salt, carrier, excipient or diluent of a composition according to the present invention is a component that is compatible with the other ingredients of the composition, in that it can be combined with the prodrugs of the present invention without eliminating the biological activity of the biologically active agent and is suitable for use with subjects as provided herein without undue adverse side effects (such as toxicity, irritation, and allergic response). Side effects are “undue” when their risk outweighs the benefit provided by the pharmaceutical composition. Examples of pharmaceutically acceptable components include, without limitation, any of the standard pharmaceutical carriers such as phosphate buffered saline solutions, water, emulsions such as oil/water emulsion, microemulsions and various types of wetting agents.

“Polyalkylene glycol” refers to straight or branched polyalkylene glycol polymers such as polyethylene glycol, polypropylene glycol, and polybutylene glycol, and includes the monoalkylether of the polyalkylene glycol. In a particular embodiment, the polyalkylene glycol is polyethylene glycol or “PEG.” The term “PEG subunit” refers to a single polyethylene glycol unit, i.e., —(CH₂CH₂O)—.

“Prodrug” refers to a biologically active agent that has been chemically derivitized such that, upon administration to a subject, the prodrug is metabolized to yield the biologically active agent.

“Solubility” refers to the tendency of one substance to blend uniformly with another.

“Substituted,” as applied to alkyl, aryl, alkylaryl, arylalkyl, and the like, refers to substituents selected from alkyl, alkenyl, heterocyclyl, cycloalkyl, aryl (including heteroaryl), alkylaryl, arylalkyl, halo (e.g., F, Cl, Br, or I), alkoxy, amine, trifluoroalkyl, such as trifluoromethyl, —CN, —NO₂, —SR′, —N₃, —C(═O)NR′₂, —NR′C(═O)R″, —C(═O)R′, —C(═O)OR′, —OC(═O)R′, —NR′SO₂R′, —OC(═O)NR′₂, —NR′C(═O)OR′, —SO₂R′, and —SO₂NR′₂, where R′ is, individually, hydrogen, alkyl, substituted alkyl, aryl, substituted aryl, arylalkyl, substituted arylalkyl, alkylaryl, or substituted alkylaryl, and r is an integer from 1 to 6. The term “substituted” as applied to alkyl, aryl (including heteroaryl), cycloalkyl and the like refers to the substituents described above.

“Treat” or “treating” as used herein refers to any type of treatment that imparts a benefit to a subject afflicted with a disease or illness, including improvement in the condition of the subject (e.g., in one or more symptoms), delay in the progression of the condition, prevention or delay of the onset of the disease or illness, etc.

5.2 Prodrug Compounds

The prodrugs, D-C(X)XR (Formula I, see below), exhibit one or more improved characteristics relative to the unconjugated biologically active moiety, D. For example, the addition of the —C(X)XR moiety can protect the biologically active moiety, D, from degradation in various environments (such as the gastrointestinal tract (GI tract)), such that less of D is degraded in the prodrug form than would be degraded in the absence of the —C(X)XR moiety in such enviroments. In particular, certain prodrugs of the invention can be orally administered in a dosage that ultimately provides a pharmaceutically acceptable amount of the biologically active moiety, D, in systemic circulation. That is to say, a sufficient amount of prodrug can survive in the GI tract and enter the bloodstream such that upon hydrolysis and release of the biologically active moiety, D, the parent biologically active moiety, D, is systemically present in a pharmaceutically acceptable amount. Preferably, the addition of the —C(X)XR moiety improves the delivery of orally administered active compound into the bloodstream for orally administered prodrug relative to the delivery of orally administered unconjugated parent biologically active moiety, D, into the bloodstream. More preferably, the improvement of the delivery of active compound into the bloodstream for orally administered prodrug is at least 2 times times the delivery of orally administered unconjugated parent biologically active moiety, D, into the bloodstream. Still more preferably, the improvement of the delivery of active compound into the bloodstream for orally administered prodrug is at least 3, 4, 5, 6, 7, 8, 9, 10, 15, 20, 25, 30, 40, 50, 60, 70, 80, 90, 100, 150, 200, 300, 400, or 500 times the delivery of orally administered unconjugated parent biologically active moiety, D, into the bloodstream. Thus, administration of the prodrug of the invention can provide greater bioavailability of the biologically active agent relative to administration of unconjugated biologically active agent, D.

Other examples of improved characteristics of the prodrug, D-C(X)XR, relative to unconjugated parent biologically active moiety, D, include improved ability of the prodrug to pass through the GI tract and enter the blood stream; improved hydrophilicity, hydrophobicity, or amphiphilicity of the prodrug; improved solubility of the prodrug in aqueous environments or organic solvents; improved ability of the prodrug to cross cell membranes; improved ability of the prodrug to traverse the blood-brain barrier; improved ability of the prodrug to target a certain receptor, cell, tissue, or organ; and improved pharmacokinetic profile of the prodrug. In a preferred embodiment, the degradation of the biologically active agent component of the prodrug is less than the degradation of unconjugated biologically active agent, D, at a pH of about 2 for less than about 2 hours. The biologically active agent component of the prodrug is more stable as a component of the prodrug than the biologically active agent from which the prodrug is derived in the presence of plasma, proteases, liver homogenate, acidic conditions and/or basic conditions.

The prodrug further can have a different level of biological activity relative to the unconjugated drug. In some embodiments, the prodrug retains some or all of the activity, but by virtue of conjugation to appropriate R groups, is less susceptible to in vivo degradation, and thus, has an increased plasma half life. In other embodiments, the prodrug has less activity than the prodrug, or no activity whatsoever, and only has activity upon hydrolysis and release of the active drug. Reduced activity can be preferred, for example, when longterm release of the drug is desirable.

In many embodiments, for example, the biologically active agent functions, in part, by binding to an active site in a receptor. Often, when a functional group, such as a hydroxyl, thiol, amine or carboxylic acid group is modified, the agent no longer binds in the active site. Depending on whether the prodrug itself is to be biologically active, or (relatively) inactive, one can design a prodrug, using the technology described herein, so that the binding to the active site is or is not retained.

In some embodiments, the prodrugs are monoconjugates. In other embodiments, the prodrugs are multi-conjugates. The number of —C(X)XR moieties on D is limited only by the number of conjugation sites on D. In still other embodiments, the prodrug compositions of the present invention are a mixture of mono- and di-conjugates. For example, in some embodiments, the biologically active agent includes or is modified to include a moiety, such as a urea or thiourea moiety, which possesses two conjugatable nitrogens. Thus, there are two NH moieties that can be conjugated to either an R group, or to a —C(O)OR group, to form the prodrugs described herein. Suitable prodrug forms include the product of monoconjugation at either nitrogen or diconjugation at both nitrogens. Moreover, the —C(X)XR moieties may themselves be conjugated to other —C(X)XR moieties.

The prodrug compounds described herein have the following formula:

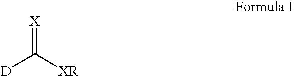

wherein:

X is O, S, or NR′,

R′is, individually, hydrogen, alkyl, substituted alkyl, aryl, substituted aryl, arylalkyl, substituted arylalkyl, alkylaryl, or substituted alkylaryl,

“substituted,” as applied to alkyl, aryl, alkylaryl, and arylalkyl, refers to substituents selected from alkyl, alkenyl, heterocyclyl, cycloalkyl, aryl (including heteroaryl), alkylaryl, arylalkyl, halo (e.g., F, Cl, Br, or I), alkoxy, amine, trifluoroalkyl, such as trifluoromethyl, —CN, —NO₂, —SR′, —N₃, —C(═O)NR′₂, —NR′C(═O)R″, —C(═O)R′, —C(═O)OR′, —OC(═O)R′, —NR′SO₂R′, —OC(═O)NR′₂, —NR′C(═O)OR′, —SO₂R′, and —SO₂NR′₂,

D is a biologically active agent (“drug”) that includes a functional group, such as a urea, thiourea, amide, thioamide, imide, thioimide, carbamate, thiocarbamate, sulfonamide, sulfonimide, phosphoramide, and the like, or which originally included a hydroxyl, amine, thiol, and/or carboxylic acid group, where such group has been modified to be in the form of a urea, thiourea, amide, thioamide, imide, thioimide, carbamate, thiocarbamate, sulfonamide, sulfonimide, phosphoramide, and the like,

the linkage between D and the C═X moiety (such as a carbonyl) is through an —N— linkage, formed from an NH group present in the urea, thiourea, amide, thioamide, imide, thioimide, carbamate, thiocarbamate, sulfonamide, sulfonimide, phosphoramide, and the like before being coupled to the C═X moiety in Formula I, resulting in functional groups that are more readily hydrolyzable than where D is directly coupled to a carbamate functional group,

and R is a “modifying moiety.”

Modifying moieties are moieties that modify the drug, and provide the prodrug with desired properties. For example, the modifying moiety can protect the biologically active moiety from degradation in various environments (such as the GI tract), such that less of the biologically active moiety is degraded in the prodrug form than would be degraded in the absence of the modifying moiety in such environments. Preferably, the addition of the modifying moiety improves the delivery of active compound into the bloodstream for orally administered prodrug relative to the delivery of orally administered unconjugated parent biologically active moiety into the bloodstream. More preferably, the improvement of the delivery of active compound into the bloodstream for orally administered prodrug is at least 2 times times the delivery of orally administered unconjugated parent biologically active moiety, into the bloodstream. Still more preferably, the improvement of the delivery of active compound into the bloodstream for orally administered prodrug is at least 3, 4, 5, 6, 7, 8, 9, 10, 15, 20, 25, 30, 40, 50, 60, 70, 80, 90, 100, 150, 200, 300, 400, or 500 times the delivery of orally administered unconjugated parent biologically active moiety, D, into the bloodstream. Thus, the modifying moiety is preferably selected to provide a prodrug that enables greater bioavailability of the biologically active agent than administration of unconjugated biologically active agent.

A subset of the compounds of Formula 1 has the following formula:

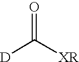

where D, X and R are as defined above. In one embodiment, X is nitrogen.

Another subset of the compounds of Formula I has the following formula:

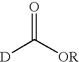

where D and R are as defined above.

Further non-limiting examples of prodrugs according to the invention include:

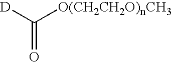

where n is greater than 2, preferably from 2 to 25, more preferably from 2 to 20, ideally from 2 to 15;

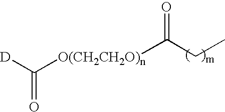

where n is at least 2 and m is from 2 to 20. Preferably n is from 2 to 25, more preferably from 2 to 20, ideally from 2 to 15. Preferably m is from 2-20, more preferably from 2 to 15, more preferably from 2 to 10; and

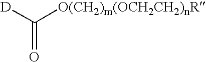

where n is at least 2 and m is from 2 to 20. Preferably n is from 2 to 25, more preferably from 2 to 20, ideally from 2 to 15. Preferably m is from 2-20, more preferably from 2 to 15, more preferably from 2 to 10. R″ is alkyl or substituted alkyl, hydroxy or methoxy. Preferably R″ is C₁₋₂₀ alkyl, more preferably C₂₋₁₅ alkyl, more preferably C₂₋₁₀ alkyl.

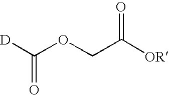

where R′ is a modifying moiety R as defined above. For example, R′ can be a straight or branched polymeric moiety comprising one or more straight or branched polyalkylene glycol moieties and/or one or more straight or branched, substituted or unsubstituted alkyl moieties. The polyalkylene glycol moieties preferably include from 2 to 25 polyalkylene glycol subunits, more preferably from 2 to 20, ideally from 2 to 15. The polyalkylene glycol moieties preferably comprise polyethylene glycol. The alkyl moieties from 2-20, more preferably from 2 to 15, more preferably from 2 to 10 carbon atoms. The alkyl moieties are preferably alkane moieties.

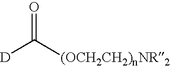

where each R″ is independently selected and the two R″ moieties include at least one modifying moiety as described herein. Representative examples of R″ include H, substituted alkyl, targeting moieties, and hydrophilic or amphiphilic polymers and/or oligomers. Examples of substituted alkyl are as defined herein, and also specifically include moieties in which the 2 R″ moieties are combined to form a heterocyclic moiety as defined herein. Preferred heterocyclic moieties include morpholynic, piperidinyl, pyrazine, and the like.

In one embodiment, one R″ is a targeting moiety. In another embodiment, one R″ is a targeting moiety and another is another modifying moiety, such as a hydrophilic or amphiphilic polymer or oligomer and may also include a salt forming moiety.

In any of the foregoing examples, D may be conjugated at multiple conjugation sites. The number of oligomers is limited only by the number of conjugation sites.

In certain embodiments, the R and D moieties can be reversed to provide a prodrug having a formula: RC(X)XD, where R, X and D are defined as above.

One aspect of the invention includes Z-C(X)—XR, where X and R are as defined as above and Z is a hydroxyl, thiol, amine, or an activating group, such as halogen, anhydride, and the like that permits activation and/or attachment to D or R.

Components D and R are described in more detail below.

5.2.1 Biologically Active Agents (“D”)

The biologically active agents may be various agents as will be understood by those skilled in the art. Representative non-limiting classes of biologically active agents useful in the present invention include those falling into the following therapeutic categories: ACE-inhibitors; anti-anginal drugs; anti-arrhythmias; anti-asthmatics; anti-cholesterolemics; anti-convulsants; anti-depressants; anti-diarrhea preparations; anti-histamines; anti-hypertensive drugs; anti-infectives; anti-inflammatory agents; anti-lipid agents; anti-manics; anti-nauseants; anti-stroke agents; anti-thyroid preparations; anti-tumor drugs; anti-tussives; anti-uricemic drugs; anti-viral agents; acne drugs; alkaloids; amino acid preparations; anabolic drugs; analgesics; anesthetics; angiogenesis inhibitors; antacids; anti-arthritics; antibiotics; anticoagulants; antiemetics; antiobesity drugs; antiparasitics; antipsychotics; antipyretics; antispasmodics; antithrombotic drugs; anxiolytic agents; appetite stimulants; appetite suppressants; beta blocking agents; bronchodilators; cardiovascular agents; cerebral dilators; chelating agents; cholecystokinin antagonists; chemotherapeutic agents; cognition activators; contraceptives; coronary dilators; cough suppressants; decongestants; deodorants; dermatological agents; diabetes agents; diuretics; emollients; enzymes; erythropoietic drugs; expectorants; fertility agents; fungicides; gastrointestinal agents; growth regulators; hormone replacement agents; hyperglycemic agents; hypnotics; hypoglycemic agents; laxatives; migraine treatments; mineral supplements; mucolytics; narcotics; neuroleptics; neuromuscular drugs; NSAIDS; nutritional additives; peripheral vasodilators; prostaglandins; psychotropics; renin inhibitors; respiratory stimulants; steroids; stimulants; sympatholytics; thyroid preparations; tranquilizers; uterine relaxants; vaginal preparations; vasoconstrictors; vasodilators; vertigo agents; vitamins; and wound healing agents. Immunotherapeutics, such as interferon-alpha (used to treat, among other disorders, hepatitis C) represent a class of drugs that can be converted to the prodrugs described herein.

Representative non-limiting examples of therapeutic agents useful in the present invention include, but are not limited to, phenyloin, droperidol, sulperidol, primidone, clonazepam, glipizide, glyburide, tolbutamide, and piperidine derivatives, such as those described in U.S. Pat. No. 5,668,151 to Poindexter et al. In particular embodiments, the biologically active agent is a piperidine derivative, such as those described in U.S. Pat. No. 5,668,151 to Poindexter et al. In other particular embodiments, the biologically active agent is diphenylhydantoin, such as DILANTIN® (phenyloin). Further classes of drugs are described in more detail below.

5.2.1.1 Hydroxy-Containing Drugs

Hydroxy-containing drugs can be modified to be in the form of the prodrugs described herein. For example, the hydroxy group can be reacted to form a carbamate moiety (i.e., —OC(O)NHR′), which includes an active NH group which can be further reacted to form an additional carbamate moiety.

Several antineoplastic anti-tumor agents, such as doxorubicin, bleomycin, vincristine (vinblastine), daunorubicin, idarubicin, and the like, include hydroxy groups. These agents are normally associated with rather severe side effects. Other antitumor compounds, such as taxol and the like, include hydroxy groups, and these can be derivatized as described herein. Some of these agents have, in the past, been incorporated into liposomes or other drug delivery devices to minimize these side effects, but according to the present invention, the agents can be converted to prodrugs and also minimize the side effects. Of course, the prodrugs can also be incorporated into liposomes and other drug delivery devices, as described in more detail herein.

Several anti-viral and/or anti-tumor nucleosides, such as ddI(didanosine), ddC (zalcitabine), d4T (stavudine), FTC, lamivudine (3TC), 1592U89 (4-[2-amino-6-(cyclopropylamino)-9H-purin-9-yl]-2-cyclopentene-1-methanol), AZT (zidovudine), DAPD (D-2,6-diaminopurine dioxolane) and F-ddA, and other L-nucleosides, include hydroxyl groups and are amenable to being converted to the prodrugs described herein.

Antiviral and anticancer nucleosides are often administered several times a day. It would be advantageous to provide these agents in a once-a-day or twice-a-day format. This can be achieved, for these drugs and for the other drugs described herein, by providing a combination of the active drug (i.e., for immediate release), along with a prodrug form of the drug, for sustained release. Side effects associated with several of these drugs can be minimized by administering them in the form of the prodrugs described herein.

Several antibiotics and antifungals, such as azithromycin, erythromycin, vancomycin, amphotericin B, and the like include hydroxyl groups, and can be converted to prodrugs using the technology described herein. Additionally, steroids, such as testosterone, estrogen, progesterone, and the like, also include free hydroxy groups that can be modified using the technology described herein to form prodrug forms of the steroids.

Hydroxy-containing immunosuppressive compounds, such as cyclosporine can also be modified as described herein.

Hydroxy-containing lipid lowering drugs, including statin drugs, such as LIPITOR™ (atorvastatin), and the like can also be modified as described herein. It is believed that such modifications will lower the liver toxicity of these drugs.

Anti-inflammatory compounds, including NSAIDs, such as ketoprofen, ibuprophen, and other profen drugs and COX-2 inhibitors such as Celebrex®, Vioxx®, Bextra®, Arava®, Pennsaid®, Mobic®, Enbrel®, Remicade®, Imuran®, Humira®, Prexige®, and Rituximab®, can also be modified as described herein. The package inserts of each of these products are incorporated herein by reference.

5.2.1.2 Amine-Containing Drugs

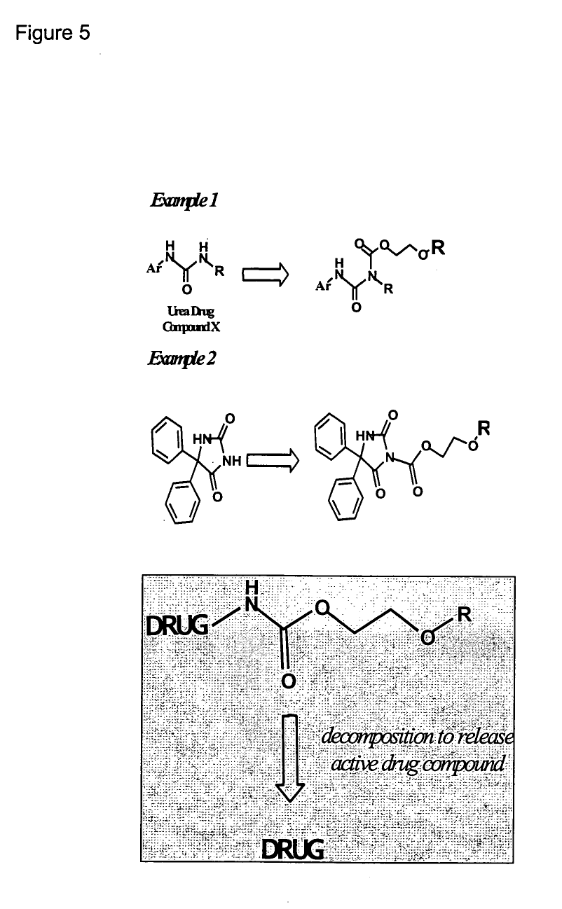

Amine-containing drugs can be converted to the prodrugs described herein. For example, the amine group can be reacted to form a carbamate moiety (i.e., —OC(O)NHR′), a urea moiety (—NHC(O)NHR′), thiourea moiety (—NHC(S)NHR′), sulfonamide moiety (—NHSO₂R′), and the like, each of which includes an active NH group which can be further reacted to form an additional carbamate moiety. This conversion, and the resulting prodrugs, are illustrated in FIG. 5.

Perhaps the most well-known amine-containing drugs are peptides and proteins, and these drugs also include a carboxylic acid group. Proteins and peptides are typically difficult to administer orally, because, among other things, they are typically broken down in the stomach and therefore have no significant bioavailability when orally administered. However, by preparing prodrug forms of these drugs, the drugs can often pass through the stomach to the intestine, where the prodrug can be hydrolyzed and release the active drug. Numerous drugs that bind to CNS receptors (i.e., antipsychotics, antidepressants, compounds used to treat neurodegenerative disorders, and the like) also include an amine functionality, and these drugs can also be converted to the prodrugs described herein.

Examples of protein and peptide drugs useful in the present invention include:

Adrenocorticotropic hormone (ACTH) peptides including, but not limited to, ACTH, human; ACTH 1-10; ACTH 1-13, human; ACTH 1-16, human; ACTH 1-17; ACTH 1-24, human; ACTH 4-10; ACTH 4-11; ACTH 6-24; ACTH 7-38, human; ACTH 18-39, human; ACTH, rat; ACTH 12-39, rat; beta-cell tropin (ACTH 22-39); biotinyl-ACTH 1-24, human; biotinyl-ACTH 7-38, human; corticostatin, human; corticostatin, rabbit; [Met(02)⁴, DLys⁸, Phe⁹] ACTH 4-9, human; [Met(0)⁴,DLys⁸, Phe⁹] ACTH 4-9, human; N-acetyl, ACTH 1-17, human; and ebiratide.

Adrenomedullin peptides including, but not limited to, adrenomedullin, adrenomedullin 1-52, human; adrenomedullin 1-12, human; adrenomedullin 13-52, human; adrenomedullin 22-52, human; pro-adrenomedullin 45-92, human; pro-adrenomedullin 153-185, human; adrenomedullin 1-52, porcine; pro-adrenomedullin (N-20), porcine; adrenomedullin 1-50, rat; adrenomedullin 11-50, rat; and proAM-N20 (proadrenomedullin N-terminal 20 peptide), rat.

Allatostatin peptides including, but not limited to, allatostatin I; allatostatin II; allatostatin III; and allatostatin IV.

Amylin peptides including, but not limited to, acetyl-amylin 8-37, human; acetylated amylin 8-37, rat; AC187 amylin antagonist; AC253 amylin antagonist; AC625 amylin antagonist; amylin 8-37, human; amylin (IAPP), cat; amylin (insulinoma or islet amyloid polypeptide(IAPP)); amylin amide, human; amylin 1-13 (diabetes-associated peptide 1-13), human; amylin 20-29 (LAPP 20-29), human; AC625 amylin antagonist; amylin 8-37, human; amylin (IAPP), cat; amylin, rat; amylin 8-37, rat; biotinyl-amylin, rat; and biotinyl-amylin amide, human. Amyloid beta-protein fragment peptides including, but not limited to, Alzheimer's disease beta-protein 12-28 (SP17); amyloid beta-protein 25-35; amyloid beta/A4-protein precursor 328-332; amyloid beta/A4 protein precursor (APP) 319-335; amyloid beta-protein 143; amyloid beta-protein 1-42; amyloid beta-protein 1-40; amyloid beta-protein 10-20; amyloid beta-protein 22-35; Alzheimer's disease beta-protein (SP28); beta-amyloid peptide 1-42, rat; beta-amyloid peptide 1-40, rat; beta-amyloid 1-11; beta-amyloid 31-35; beta-amyloid 32-35; beta-amyloid 35-25; beta-amyloid/A4 protein precursor 96-110; beta-amyloid precursor protein 657-676; beta-amyloid 1-38; [Gln¹¹]-Alzheimer's disease beta-protein; [Gln¹¹]-beta-amyloid 1-40; [Gln²²]-beta-amyloid 6-40; non-A beta component of Alzheimer's disease amyloid (NAC); P3, (A beta 17-40) Alzheimer's disease amyloid β-peptide; and SAP (serum amyloid P component) 194-204.

Angiotensin peptides including, but not limited to, A-779; Ala-Pro-Gly-angiotensin II; [Ile³, Val⁵]-angiotensin II; angiotensin III antipeptide; angiogenin fragment 108-122; angiogenin fragment 108-123; angiotensin I converting enzyme inhibitor; angiotensin I, human; angiotensin I converting enzyme substrate; angiotensin 11-7, human; angiopeptin; angiotensin I, human; angiotensin II antipeptide; angiotensin II 1-4, human; angiotensin II 3-8, human; angiotensin II 4-8, human; angiotensin II 5-8, human; angiotensin HI ([Des-Asp¹]-angiotensin II), human; angiotensin III inhibitor ([Ile⁷]-angiotensin III); angiotensin-converting enzyme inhibitor (Neothunnus macropterus); [Asn¹, Val⁵]-angiotensin I, goosefish; [Asn¹, Val⁵, Asn⁹]-angiotensin I, salmon; [Asn¹, Val⁵, Gly⁹]-angiotensin I, eel; [Asn¹, Val⁵]-angiotensin I 1-7, eel, goosefish, salmon; [Asn¹, Val⁵]-angiotensin II; biotinyl-angiotensin I, human; biotinyl-angiotensin II, human; biotinyl-Ala-Ala-Ala-angiotensin II; [Des-Asp¹]-angiotensin I, human; [p-aminophenylalanine⁶]-angiotensin II; renin substrate (angiotensinogen 1-13), human; preangiotensinogen 1-14 (renin substrate tetradecapeptide), human; renin substrate tetradecapeptide (angiotensinogen 1-14), porcine; [Sar¹]-angiotensin II, [Sar¹]-angiotensin II 1-7 amide; [Sar¹, Ala⁸]-angiotensin II; [Sar¹, Ile⁸]-angiotensin II; [Sar¹, Thr⁸]-angiotensin II; [Sar¹, Tyr(Me)⁴]-angiotensin II (Sarmesin); [Sar¹, Val⁵, Ala⁸]-angiotensin I; [Sar¹, Ile⁷]-angiotensin III; synthetic tetradecapeptide renin substrate (No. 2); [Val⁴]-angiotensin III; [Val⁵]-angiotensin II; [Val⁵]-angiotensin I, human; [Val⁵]-angiotensin I; [Val⁵, Asn¹]-angiotensin I, bullfrog; and [Val⁵, Ser⁹]-angiotensin I, fowl.

Antibiotic peptides including, but not limited to, Ac-SQNY; bactenecin, bovine; CAP 37 (20-44); carbormethoxycarbonyl-DPro-DPhe-OBzl; CD36 peptide P 139-155; CD36 peptide P 93-110; cecropin A-melittin hybrid peptide [CA(1-7)M(2-9)NH2]; cecropin B, free acid; CYS(Bzl)₈₄ CD fragment 81-92; defensin (human) HNP-2; dermaseptin; immunostimulating peptide, human; lactoferricin, bovine (BLFC); and magainin spacer.

Antigenic polypeptides, which can elicit an enhanced immune response, enhance an immune response and or cause an immunizingly effective response to diseases and/or disease causing agents including, but not limited to, adenoviruses; anthrax; Bordetella pertussus; botulism; bovine rhinotracheitis; Branhamella catarrhalis; canine hepatitis; canine distemper; Chlamydiae; cholera; coccidiomycosis; cowpox; cytomegalovirus; Dengue fever; dengue toxoplasmosis; diphtheria; encephalitis; enterotoxigenic E. coli; Epstein Barr virus; equine encephalitis; equine infectious anemia; equine influenza; equine pneumonia; equine rhinovirus; Escherichia coli; feline leukemia; flavivirus; globulin; haemophilus influenza type b; Haemophilus influenzae; Haemophilus pertussis; Helicobacter pylon; hemophilus; hepatitis; hepatitis A; hepatitis B; Hepatitis C; herpes viruses; HIV; HIV-1 viruses; HIV-2 viruses; HTLV; influenza; Japanese encephalitis; Klebsiellae species; Legionella pneumophila; leishmania; leprosy; lyme disease; malaria immunogen; measles; meningitis; meningococcal; Meningococcal polysaccharide group A; Meningococcal polysaccharide group C; mumps; mumps virus; mycobacteria; Mycobacterium tuberculosis; Neisseria; Neisseria gonorrhea; Neisseria meningitidis; ovine blue tongue; ovine encephalitis; papilloma; parainfluenza; paramyxoviruses; Pertussis; plague; pneumococcus; Pneumocystis carinii; pneumonia; poliovirus; proteus species; Pseudomonas aeruginosa; rabies; respiratory syncytial virus; rotavirus; rubella; salmonellae; schistosomiasis; shigellae; simian immunodeficiency virus; smallpox; Staphylococcus aureus; Staphylococcus species; Streptococcus pneumonia; Streptococcus pyogenes; Streptococcus species; swine influenza; tetanus; Treponema pallidum; typhoid; vaccinia; varicella-zoster virus; and vibrio cholerae.

Anti-microbial peptides including, but not limited to, buforin I; buforin II; cecropin A; cecropin B; cecropin P1, porcine; gaegurin 2 (Rana rugosa); gaegurin 5 (Rana rugosa); indolicidin; protegrin-(PG)-I; magainin 1; and magainin 2; and T-22 [Tyr^{5,12}, Lys⁷]-poly-phemusin II peptide.

Apoptosis related peptides including, but not limited to, Alzheimer's disease beta-protein (SP28); calpain inhibitor peptide; capsase-1 inhibitor V; capsase-3, substrate IV; caspase-1 inhibitor I, cell-permeable; caspase-1 inhibitor VI; caspase-3 substrate III, fluorogenic; caspase-I substrate V, fluorogenic; caspase-3 inhibitor I, cell-permeable; caspase-6 ICE inhibitor III; [Des-Ac, biotin]-ICE inhibitor III; IL-1 B converting enzyme (ICE) inhibitor II; IL-1 B converting enzyme (ICE) substrate IV; MDL 28170; and MG-132.

Atrial natriuretic peptides including, but not limited to, alpha-ANP (alpha-chANP), chicken; anantin; ANP 1-11, rat; ANP 8-30, frog; ANP 11-30, frog; ANP-21 (fANP-21), frog; ANP-24 (fANP-24), frog; ANP-30, frog; ANP fragment 5-28, human, canine; ANP-7-23, human; ANP fragment 7-28, human, canine; alpha-atrial natriuretic polypeptide 1-28, human, canine; A71915, rat; atrial natriuretic factor 8-33, rat; atrial natriuretic polypeptide 3-28, human; atrial natriuretic polypeptide 4-28, human, canine; atrial natriuretic polypeptide 5-27; human; atrial natriuretic aeptide (ANP), eel; atriopeptin I, rat, rabbit, mouse; atriopeptin II, rat, rabbit, mouse; atriopeptin III, rat, rabbit, mouse; atrial natriuretic factor (rANF), rat, auriculin A (rat ANF 126-149); auriculin B (rat ANF 126-150); beta-ANP (1-28, dimer, antiparallel); beta-rANF 17-48; biotinyl-alpha-ANP 1-28, human, canine; biotinyl-atrial natriuretic factor (biotinyl-rANF), rat; cardiodilatin 1-16, human; C-ANF 4-23, rat; Des-[Cys¹⁰⁵, Cys¹²¹]-atrial natriuretic factor 104-126, rat; [Met(O)¹²] ANP 1-28, human; [Mpr⁷,DAla⁹]ANP 7-28, amide, rat; prepro-ANF 104-116, human; prepro-ANF 26-55 (proANF 1-30), human; prepro-ANF 56-92 (proANF 31-67), human; prepro-ANF 104-123, human; [Tyr°]-atriopeptin I, rat, rabbit, mouse; [Tyr⁰]-atriopeptin II, rat, rabbit, mouse; [Tyr⁰]-prepro ANF 104-123, human; urodilatin (CDD/ANP 95-126); ventricular natriuretic peptide (VNP), eel; and ventricular natriuretic peptide (VNP), rainbow trout.

Bag cell peptides including, but not limited to, alpha bag cell peptide; alpha-bag cell peptide 1-9; alpha-bag cell peptide 1-8; alpha-bag cell peptide 1-7; beta-bag cell factor; and gamma-bag cell factor.

Bombesin peptides including, but not limited to, alpha-sl casein 101-123 (bovine milk); biotinyl-bombesin; bombesin 8-14; bombesin; [Leu¹³-psi (CH2NH)Leu¹⁴]-bombesin; [D-Phe⁶, Des-Met¹⁴]-bombesin 6-14 ethylamide; [DPhe¹²] bombesin; [DPhe¹²,Leu¹⁴]-bombesin; [Tyr⁴]-bombesin; and [Tyr⁴,DPhe¹²]-bombesin.

Bone GLA peptides (BGP) including, but not limited to, bone GLA protein; bone GLA protein 45-49; [Glu¹⁷, Gla^{21,24}]-osteocalcin 1-49, human; myclopeptide -2 (MP-2); osteocalcin 1-49 human; osteocalcin 37-49; human; and [Tyr³⁸, Phe^{42,46}] bone GLA protein 3849, human.

Bradykinin peptides including, but not limited to, [Ala^{2,6}, des-Pro³]-bradykinin; bradykinin; bradykinin (Bowfin. Gar); bradykinin potentiating peptide; bradykinin 1-3; bradykinin 1-5; bradykinin 1-6; bradykinin 1-7; bradykinin 2-7; bradykinin 2-9; [DPhe⁷] bradykinin; [Des-Arg⁹]-bradykinin; [Des-Arg¹⁰]-Lys-bradykinin ([Des-Arg¹⁰]-kallidin); [D-N-Me-Phe⁷]-bradykinin; [Des-Arg⁹, Leu⁸]-bradykinin; Lys-bradykinin (kallidin); Lys-[Des-Arg⁹,Leu⁸]-bradykinin ([Des-Arg¹⁰,Leu⁹]-kallidin); [Lys⁰-Hyp³]-bradykinin; ovokinin; [Lys⁰, Ala³]-bradykinin; Met-Lys-bradykinin; peptide K12 bradykinin potentiating peptide; [(pCl)Phe^{5,8}]-bradykinin; T-kinin (Ile-Ser-bradykinin); [Thi^{5,8}, D-Phe⁷]-bradykinin; [Tyr⁰]-bradykinin; [Tyr⁵]-bradykinin; [Tyr⁸]-bradykinin; and kallikrein.

Brain natriuretic peptides (BNP) including, but not limited to, BNP 32, canine; BNP-like Peptide, eel; BNP-32, human; BNP45, mouse; BNP-26, porcine; BNP-32, porcine; biotinyl-BNP-32, porcine; BNP-32, rat; biotinyl-BNP-32, rat; BNP45 (BNP 51-95, 5K cardiac natriuretic peptide), rat; and [Tyr⁰]-BNP 1-32, human.

C-peptides including, but not limited to, C-peptide; and [Tyr⁰]-C-peptide, human.

C-type natriuretic peptides (CNP) including, but not limited to, C-type natriuretic peptide, chicken; C-type natriuretic peptide-22 (CNP-22), porcine, rat, human; C-type natriuretic peptide-53 (CNP-53), human; C-type natriuretic peptide-53 (CNP-53), porcine, rat; C-type natriuretic peptide-53 (porcine, rat) 1-29 (CNP-53 1-29); prepro-CNP 1-27, rat; prepro-CNP 30-50, porcine, rat; vasonatrin peptide (VNP); and [Tyr⁰]-C-type natriuretic peptide-22 ([Tyr⁰]-CNP-22).

Calcitonin peptides including, but not limited to, biotinyl-calcitonin, human; biotinyl-calcitonin, rat; biotinyl-calcitonin, salmon; calcitonin, chicken; calcitonin, eel; calcitonin, human; calcitonin, porcine; calcitonin, rat; calcitonin, salmon; calcitonin 1-7, human; calcitonin 8-32, salmon; katacalcin (PDN-21) (C-procalcitonin); and N-proCT (amino-terminal procalcitonin cleavage peptide), human.

Calcitonin gene related peptides (CGRP) including, but not limited to, acetyl-alpha-CGRP 19-37, human; alpha-CGRP 19-37, human; alpha-CGRP 23-37, human; biotinyl-CGRP, human; biotinyl-CGRP II, human; biotinyl-CGRP, rat; beta-CGRP, rat; biotinyl-beta-CGRP, rat; CGRP, rat; CGRP, human; calcitonin C-terminal adjacent peptide; CGRP 1-19, human; CGRP 20-37, human; CGRP 8-37, human; CGRP II, human; CGRP, rat; CGRP 8-37, rat; CGRP 29-37, rat; CGRP 30-37, rat; CGRP 31-37, rat; CGRP 32-37, rat; CGRP 33-37, rat; CGRP 31-37, rat; ([Cys(Acm)^{2,7}]-CGRP; elcatonin; [Tyr⁰]-CGRP, human; [Tyr⁰]-CGRP II, human; [Tyr⁰]-CGRP 28-37, rat; [Tyr⁰]-CGRP, rat; and [Tyr²²]-CGRP 22-37, rat.

CART peptides including, but not limited to, CART, human; CART 55-102, human; CART, rat; and CART 55-102, rat.

Casomorphin peptides including, but not limited to, beta-casomorphin, human; beta-casomorphin 1-3; beta-casomorphin 1-3, amide; beta-casomorphin, bovine; beta-casomorphin 14, bovine; beta-casomorphin 1-5, bovine; beta-casomorphin 1-5, amide, bovine; beta-casomorphin 1-6, bovine; [DAla²]-beta-casomorphin 1-3, amide, bovine; [DAla²,Hyp⁴,Tyr⁵]-beta-casomorphin 1-5 amide; [DAla²,DPro⁴,Tyr⁵]-beta-casomorphin 1-5, amide; [DAla²,Tyr⁵]-beta-casomorphin 1-5, amide, bovine; [DAla^{2,4},Tyr⁵]-beta-casomorphin 1-5, amide, bovine; [DAla², (pCl)Phe³]-beta-casomorphin, amide, bovine; [DAla²]-beta-casomorphin 14, amide, bovine; [DAla²]-beta-casomorphin 1-5, bovine; [DAla²]-beta-casomorphin 1-5, amide, bovine; [DAla²,Met⁵]-beta-casomorphin 1-5, bovine; [DPro²]-beta-casomorphin 1-5, amide, bovine; [DAla²]-beta-casomorphin 1-6, bovine; [[)Pro²]-beta-casomorphin 14, amide; [Des-Tyr¹]-beta-casomorphin, bovine; [DAla²4,Tyr⁵]-beta-casomorphin 1-5, amide, bovine; [DAla², (pCl)Phe³]-beta-casomorphin, amide, bovine; [DAla²]-beta-casomorphin 14, amide, bovine; [DAla²]-beta-casomorphin 1-5, bovine; [DAla²]-beta-casomorphin 1-5, amide, bovine; [DAla²,Met⁵]-beta-casomorphin 1-5, bovine; [DPro²]-beta-casomorphin 1-5, amide, bovine; [DAla²]-beta-casomorphin 1-6, bovine; [DPro²]-beta-casomorphin 14, amide; (Des-Tyr¹]-beta-casomorphin, bovine; and [Val³]-beta-casomorphin 14, amide, bovine.

Chemotactic peptides including, but not limited to, defensin I (human) HNP-1 (human neutrophil peptide-1); and N-formyl-Met-Leu-Phe.

Cholecystokinin (CCK) peptides including, but not limited to, caerulein; cholecystokinin; cholecystokinin-pancreozymin; CCK-33, human; cholecystokinin octapeptide 14 (non-sulfated) (CCK 26-29, unsulfated); cholecystokinin octapeptide (CCK 26-33); cholecystokinin octapeptide (non-sulfated) (CCK 26-33, unsulfated); cholecystokinin heptapeptide (CCK 27-33); cholecystokinin tetrapeptide (CCK 30-33); CCK-33, porcine; CR 1 409, cholecystokinin antagonist; CCK flanking peptide (unsulfated); N-acetyl cholecystokinin, CCK 26-30, sulfated; N-acetyl cholecystokinin, CCK 26-31, sulfated; N-acetyl cholecystokinin, CCK 26-31, non-sulfated; prepro CCK fragment V-9-M; and proglumide.

Colony-stimulating factor peptides including, but not limited to, colony-stimulating factor (CSF); GMCSF; MCSF; and G-CSF.

Corticortropin releasing factor (CRF) peptides including, but not limited to, astressin; alpha-helical CRF 1241; biotinyl-CRF, ovine; biotinyl-CRF, human, rat; CRF, bovine; CRF, human, rat; CRF, ovine; CRF, porcine; [Cys²¹]-CRF, human, rat; CRF antagonist (alpha-helical CRF 9-41); CRF 6-33, human, rat; [DPro⁵]-CRF, human, rat; [D-Phe¹², Nle^{21,38}]-CRF 12-41, human, rat; eosinophilotactic peptide; [Met(0)²¹]-CRF, ovine; [Nle²¹ Tyr³²]-CRF, ovine; prepro CRF 125-151, human; sauvagine, frog; [Tyr⁰]-CRF, human, rat; [Tyr⁰]-CRF, ovine; [Tyr⁰]-CRF 3441, ovine; [Tyr⁰]-urocortin; urocortin amide, human; urocortin, rat; urotensin I (Catostomus commersoni); urotensin II; and urotensin II (Rana ridibunda).

Cortistatin peptides including, but not limited to, cortistatin 29; cortistatin 29 (1-13); [Tyr⁰]-cortistatin 29; pro-cortistatin 2847; and pro-cortistatin 51-81.

Cytokine peptides including, but not limited to, tumor necrosis factor; and tumor necrosis factor-β (TNF-β).

Dermorphin peptides including, but not limited to, dermorphin and dermorphin analog 1-4.

Dynorphin peptides including, but not limited to, big dynorphin (prodynorphin 209-240), porcine; biotinyl-dynorphin A (biotinyl-prodynorphin 209-225); [DAla², DArg⁶]-dynorphin A 1-13, porcine; [D-Ala²]-dynorphin A, porcine; [D-Ala²]-dynorphin A amide, porcine; [D-Ala²]-dynorphin A 1-13, amide, porcine; [D-Ala²]-dynorphin A 1-9, porcine; [DArg⁶]-dynorphin A 1-13, porcine; [DArg⁸]-dynorphin A 1-13, porcine; [Des-Tyr¹]-dynorphin A 1-8; [D-Pro¹⁰]-dynorphin A 1-11, porcine; dynorphin A amide, porcine; dynorphin A 1-6, porcine; dynorphin A 1-7, porcine; dynorphin A 1-8, porcine; dynorphin A 1-9, porcine; dynorphin A 1-10, porcine; dynorphin A 1-10 amide, porcine; dynorphin A 1-11, porcine; dynorphin A 1-12, porcine; dynorphin A 1-13, porcine; dynorphin A 1-13 amide, porcine; DAKLI (dynorphin A-analogue kappa ligand); DAKLI-biotin ([Arg^{11,13}]-dynorphin A (1-13)-Gly-NH(CH2)5NH-biotin); dynorphin A 2-17, porcine; dynorphin 2-17, amide, porcine; dynorphin A 2-12, porcine; dynorphin A 3-17, amide, porcine; dynorphin A 3-8, porcine; dynorphin A 3-13, porcine; dynorphin A 3-17, porcine; dynorphin A 7-17, porcine; dynorphin A 8-17, porcine; dynorphin A 6-17, porcine; dynorphin A 13-17, porcine; dynorphin A (prodynorphin 209-225), porcine; dynorphin B 1-9; [MeTyr¹, MeArg⁷, D-Leu⁸]-dynorphin 1-8 ethyl amide; [(nMe)Tyr¹] dynorphin A 1-13, amide, porcine; [Phe⁷]-dynorphin A 1-7, porcine; [Phe⁷]-dynorphin A 1-7, amide, porcine; and prodynorphin 228-256 (dynorphin B 29) (leumorphin), porcine.

Endorphin peptides including, but not limited to, alpha-neo-endorphin, porcine; beta-neo-endorphin; Ac-beta-endorphin, camel, bovine, ovine; Ac-beta-endorphin 1-27, camel, bovine, ovine; Ac-beta-endorphin, human; Ac-beta-endorphin 1-26, human; Ac-beta-endorphin 1-27, human; Ac-gamma-endorphin (Ac-beta-lipotropin 61-77); acetyl-alpha-endorphin; alpha-endorphin (beta-lipotropin 61-76); alpha-neo-endorphin analog; alpha-neo-endorphin 1-7; [Arg⁸]-alpha-neo-endorphin 1-8; beta-endorphin (beta-lipotropin 61-91), camel, bovine, ovine; beta-endorphin 1-27, camel, bovine, ovine; beta-endorphin, equine; beta-endorphin (beta-lipotropin 61-91), human; beta-endorphin (1-5)+(16-31), human; beta-endorphin 1-26, human; beta-endorphin 1-27, human; beta-endorphin 6-31, human; beta-endorphin 18-31, human; beta-endorphin, porcine; beta-endorphin, rat; beta-lipotropin 1-10, porcine; beta-lipotropin 60-65; beta-lipotropin 61-64; beta-lipotropin 61-69; beta-lipotropin 88-91; biotinyl-beta-endorphin (biotinyl-beta-lipotropin 61-91); biocytin-beta-endorphin, human; gamma-endorphin (beta-lipotropin 61-77); [DAla²]-alpha-neo-endorphin 1-2, amide; [DAla²]-beta-lipotropin 61-69; [DAla²]-gamma-endorphin; [Des-Tyr¹]-beta-endorphin, human; [Des-Tyr¹]-gamma-endorphin (beta-lipotropin 62-77); [Leu⁵]-beta-endorphin, camel, bovine, ovine; [Met⁵, Lys⁶]-alpha-neo-endorphin 1-6; [Met⁵, Lys^{6,7}]-alpha-neo-endorphin 1-7; and [Met⁵, Lys⁶, Arg⁷]-alpha-neo-endorphin 1-7.

Endothelin peptides including, but not limited to, endothelin-1 (ET-1); endothelin-1[Biotin-Lys⁹]; endothelin-1 (1-15), human; endothelin-1 (1-15), amide, human; Ac-endothelin-1 (16-21), human; Ac-[DTrp¹⁶]-endothelin-1 (16-21), human; [Ala^{3,11}]ndothelin-1; [Dpr¹, Asp¹⁵]-endothelin-1; [Ala²]-endothelin-3, human; [Ala¹⁸]-endothelin-1, human; [Asn¹⁸]-endothelin-1, human; [Res-701-1]-endothelin B receptor antagonist; Suc-[Glu⁹, Ala ^{11,15}]-endothelin-1 (8-21), IRL-1620; endothelin-C-terminal hexapeptide; [D-Val²²]-big endothelin-1 (16-38), human; endothelin-2 (ET-2), human, canine; endothelin-3 (ET-3), human, rat, porcine, rabbit; biotinyl-endothelin-3 (biotinyl-ET-3); prepro-endothelin-1 (94-109), porcine; BQ-518; BQ-610; BQ-788; endothelium-dependent relaxation antagonist; FR139317; IRL-1038; JKC-30 1; JKC-302; PD-145065; PD 142893; sarafotoxin S6a (atractaspis engaddensis); sarafotoxin S6b (atractaspis engaddensis); sarafotoxin S6c (atractaspis engaddensis); [Lys⁴]-sarafotoxin S6c; sarafotoxin S6d; big endothelin-1, human; biotinyl-big endothelin-1, human; big endothelin-1 (1-39), porcine; big endothelin-3 (2241), amide, human; big endothelin-1 (22-39), rat; big endothelin-1 (1-39), bovine; big endothelin-1 (22-39), bovine; big endothelin-1 (19-38), human; big endothelin-1 (22-38), human; big endothelin-2, human; big endothelin-2 (22-37), human; big endothelin-3, human; big endothelin-1, porcine; big endothelin-1 (22-39) (prepro-endothelin-1 (74-91)); big endothelin-1, rat; big endothelin-2 (1-38), human; big endothelin-2 (22-38), human; big endothelin-3, rat; biotinyl-big endothelin-1, human; and [Tyr¹²³]-prepro-endothelin (110-130), amide, human.

ETa receptor antagonist peptides including, but not limited to, [BQ-123]; [BE18257B]; [BE-18257A]/[W-7338A]; [BQ-485]; FR139317; PD-151242; and TTA-386.

ETh receptor antagonist peptides including, but not limited to, [BQ-3020]; [RES-701-3]; and [IRL-1720]

Enkephalin peptides including, but not limited to, adrenorphin, free acid; amidorphin (proenkephalin A (104-129)-NH2), bovine; BAM-12P (bovine adrenal medulla dodecapeptide); BAM-22P (bovine adrenal medulla docosapeptide); benzoyl-Phe-Ala-Arg; enkephalin; [D-Ala², D-Leu⁵]-enkephalin; [D-Ala², D-Met⁵]-enkephalin; [DAla²]-Leu-enkephalin, amide; [DAla²,Leu⁵,Arg⁶]-enkephalin; [Des-Tyr¹,DPen²⁵]-enkephalin; [Des-Tyr¹,DPen²,Pen⁵]-enkephalin; [Des-Tyr¹]-Leu-enkephalin; [1-Pen^{2,5}]-enkephalin; [DPen², Pen⁵]-enkephalin; enkephalinase substrate; [D-Pen², pCI-Phe⁴, D-Pen⁵]-enkephalin; Leu-enkephalin; Leu-enkephalin, amide; biotinyl-Leu-enkephalin; [D-Ala²]-Leu-enkephalin; [D-Ser²]-Leu-enkephalin-Thr (delta-receptor peptide) (DSLET); [D-Thr²]-Leu-enkephalin-Thr (DTLET); [Lys⁶]-Leu-enkephalin; [Met⁵,Arg⁶]-enkephalin; [Met⁵,Arg⁶]-enkephalin-Arg; [Met⁵,Arg⁶,Phe⁷]-enkephalin, amide; Met-enkephalin; biotinyl-Met-enkephalin; [D-Ala²]-Met-enkephalin; [D-Ala²]-Met-enkephalin, amide; Met-enkephalin-Arg-Phe; Met-enkephalin, amide; [Ala²]-Met-enkephalin, amide; [DMet2,Pro⁵]-enkephalin, amide; [DTrp²]-Met-enkephalin, amide, metorphinamide (adrenorphin); peptide B, bovine; 3200-Dalton adrenal peptide E, bovine; peptide F, bovine; preproenkephalin B 186-204, human; spinorphin, bovine; and thiorphan (D, L, 3-mercapto-2-benzylpropanoyl-glycine).

Fibronectin peptides including, but not limited to platelet factor-4 (58-70), human; echistatin (Echis carinatus); E, P, L selectin conserved region; fibronectin analog; fibronectin-binding protein; fibrinopeptide A, human; [Tyr⁰]-fibrinopeptide A, human; fibrinopeptide B, human; [Glu¹]-fibrinopeptide B, human; [Tyr¹⁵]-fibrinopeptide B, human; fibrinogen beta-chain fragment of 24-42; fibrinogen binding inhibitor peptide; fibronectin related peptide (collagen binding fragment); fibrinolysis inhibiting factor; FN-C/H-1 (fibronectin heparin-binding fragment); FN-C/H-V (fibronectin heparin-binding fragment); heparin-binding peptide; laminin penta peptide, amide; Leu-Asp-Val-NH2 (LDV-NH2), human, bovine, rat, chicken; necrofibrin, human; necrofibrin, rat; and platelet membrane glycoprotein IIB peptide 296-306.

Galanin peptides including, but not limited to, galanin, human; galanin 1-19, human; preprogalanin 1-30, human; preprogalanin 65-88, human; preprogalanin 89-123, human; galanin, porcine; galanin 1-16, porcine, rat; galanin, rat; biotinyl-galanin, rat; preprogalanin 28-67, rat; galanin 1-13-bradykinin 2-9, amide; M40, galanin 1-13-Pro-Pro-(Ala-Leu) 2-Ala-amide; C7, galanin 1-13-spantide-amide; GMAP 1-41, amide; GMAP 16-41, amide; GMAP 25-41, amide; galantide; and entero-kassinin.

Gastrin peptides including, but not limited to, gastrin, chicken; gastric inhibitory peptide (GIP), human; gastrin I, human; biotinyl-gastrin I, human; big gastrin-1, human; gastrin releasing peptide, human; gastrin releasing peptide 1-16, human; gastric inhibitory polypeptide (GIP), porcine; gastrin releasing peptide, porcine; biotinyl-gastrin releasing peptide, porcine; gastrin releasing peptide 14-27, porcine, human; little gastrin, rat; pentagastrin; gastric inhibitory peptide 1-30, porcine; gastric inhibitory peptide 1-30, amide, porcine; [Tyr⁰]-gastric inhibitory peptide 23-42, human; and gastric inhibitory peptide, rat.

Glucagon peptides including, but not limited to, [Des-His¹,Glu⁹]-glucagon, extendin-4, glucagon, human; biotinyl-glucagon, human; glucagon 19-29, human; glucagon 22-29, human; Des-His¹-[Glu⁹]-glucagon, amide; glucagon-like peptide 1, amide (preproglucagon 72-107, amide); glucagon-like peptide 1 (preproglucagon 72-108), human; glucagon-like peptide 1 (7-36) (preproglucagon 78-107, amide); glucagon-like peptide II, rat; biotinyl-glucagon-like peptide-1 (7-36) (biotinyl-preproglucagon 78-107, amide); glucagon-like peptide 2 (preproglucagon 126-159), human; oxyntomodulin/glucagon 37; and valosin (peptide VQY), porcine.

Gn-RH associated peptides (GAP) including, but not limited to, Gn-RH associated peptide 25-53, human; Gn-RH associated peptide 1-24, human; Gn-RH associated peptide 1-13, human; Gn-RH associated peptide 1-13, rat; gonadotropin releasing peptide, follicular, human;[Tyr⁰]-GAP ([Tyr⁰]-Gn-RH Precursor Peptide 14-69), human; and proopiomelanocortin (POMC) precursor 27-52, porcine.

Growth factor peptides including, but not limited to, cell growth factors; epidermal growth factors; tumor growth factor; alpha-TGF; beta-TF; alpha-TGF 34-43, rat; EGF, human; acidic fibroblast growth factor; basic fibroblast growth factor; basic fibroblast growth factor 13-18; basic fibroblast growth factor 120-125; brain derived acidic fibroblast growth factor 1-11; brain derived basic fibroblast growth factor 1-24; brain derived acidic fibroblast growth factor 102-111; [Cys(Acm^{20,31})]-epidermal growth factor 20-31; epidermal growth factor receptor peptide 985-996; insulin-like growth factor (IGF)-I, chicken; IGF-I, rat; IGF-I, human; Des (1-3) IGF-1, human; R3 IGF-I, human; R3 IGF-I, human; long R3 IGF-I, human; adjuvant peptide analog; anorexigenic peptide; Des (1-6) IGF-II, human; R6 IGF-II, human; IGF-I analogue;IGF 1 (24-41); IGF 1 (57-70); IGF I (30-41); IGF II; IGF II (33-40); [Tyr⁰]-IGF II (33-40); liver cell growth factor; midkine; midkine 60-121, human; N-acetyl, alpha-TGF 34-43, methyl ester, rat; nerve growth factor (NGF), mouse; platelet-derived growth factor; platelet-derived growth factor antagonist; transforming growth factor-alpha, human; and transforming growth factor-I, rat.

Growth hormone peptides including, but not limited to, growth hormone (hGH), human; growth hormone 143, human; growth hormone 6-13, human; growth hormone releasing factor, human; growth hormone releasing factor, bovine; growth hormone releasing factor, porcine; growth hormone releasing factor 1-29, amide, rat; growth hormone pro-releasing factor, human; biotinyl-growth hormone releasing factor, human; growth hormone releasing factor 1-29, amide, human; [D-Ala²]-growth hormone releasing factor 1-29, amide, human; [N-Ac-Tyr¹, D-Arg²]-GRF 1-29, amide; [His¹, Nle²⁷]-growth hormone releasing factor 1-32, amide; growth hormone releasing factor 1-37, human; growth hormone releasing factor 1-40, human; growth hormone releasing factor 1-40, amide, human; growth hormone releasing factor 30-44, amide, human; growth hormone releasing factor, mouse; growth hormone releasing factor, ovine; growth hormone releasing factor, rat; biotinyl-growth hormone releasing factor, rat; GHRP-6 ([His¹, Lys⁶]-GHRP); hexarelin (growth hormone releasing hexapeptide); and [D-Lys³]-GHRP-6.

GTP-binding protein fragment peptides including, but not limited to, [Arg⁸]-GTP-binding protein fragment, Gs alpha; GTP-binding protein fragment, G beta; GTP-binding protein fragment, GAlpha; GTP-binding protein fragment, Go Alpha; GTP-binding protein fragment, Gs Alpha; and GTP-binding protein fragment, G Alpha i2.

Guanylin peptides including, but not limited to, guanylin, human; guanylin, rat; and uroguanylin.

Inhibin peptides including, but not limited to, inhibin, bovine; inhibin, alpha-subunit 1-32, human; [Tyr⁰]-inhibin, alpha-subunit 1-32, human; seminal plasma inhibin-like peptide, human; [Tyr⁰]-seminal plasma inhibin-like peptide, human; inhibin, alpha-subunit 1-32, porcine; and [Tyr⁰]-inhibin, alpha-subunit 1-32, porcine.

Interferon peptides including, but not limited to, alpha interferon species (e.g., alpha1, alpha2, alpha2a, alpha2b, alpha2c, alpha2d, alpha3, alpha4, alpha4a, alpha4b, alpha5, alpha6, alpha74, alpha76, alphaA, alphaB, alphaC,, alphaC1, alphaD, alphaE, alphaF, alphaG, alphaG, alphaH, alphaI, alphaJ1, alphaJ2, alphaK, alphaL); interferon beta species (e.g., beta1a); interferon gamma species (e.g., gamma1a, gamma1b); interferon epsilon; interferon tau; interferon omega or any analogues of interferon omega. Various analogs of gamma interferon are described in Pechenov et al. “Methods for preparation of recombinant cytokine proteins V. mutant analogues of human interferon-gamma with higher stability and activity” Protein Expr. Purif 24:173-180 (2002), which is incorporated herein by reference in its entirety for teachings directed to preparation and testing of interferon analogues.

Insulin peptides including, but not limited to, insulin, human; insulin, porcine; IGF-I, human; insulin-like growth factor II (69-84); pro-insulin-like growth factor II (68-102), human; pro-insulin-like growth factor II (105-128), human; [Asp^{B28}]-insulin, human; [Lys^{B28}]-insulin, human; [Leu^{B28}] insulin, human; [Val^{B28}-insulin, human; [Ala^{B28}-insulin, human; [Asp^{B28}, Pro^{B29}]-insulin, human; [Lys^{B28}, Pro^{B29}]-insulin, human; [Leu^{B28}, Pro^{B29}]-insulin, human; [Val^{B28}, Pro^{B29}]-insulin, human; [Ala^{B28}, Pro^{B29}]-insulin, human; [Gly^{A21}]-insulin, human; [Gly^{A21} Gln^{B3}]-insulin, human; [Ala^{A21}]-insulin, human; [Ala^{A1}] GlnB³]-insulin, human; [GlnB³]-insulin, human; [GlnB³⁰]-insulin, human; [Gly^{A21} Glu^{B30}]-insulin, human; [Gly^{A21} Gln^{B3} Glu^{B30}]-insulin, human; [GlnB³ GluB³⁰]-insulin, human; B22-B30 insulin, human; B23-B30 insulin, human; B25-B30 insulin, human; B26-B30 insulin, human; B27-B30 insulin, human; B29-B30 insulin, human; the A chain of human insulin, and the B chain of human insulin.

Interleukin peptides including, but not limited to, interleukin-1 beta 165-181, rat; and interleukin-8 (IL-8, CINC/gro), rat.

Laminin peptides including, but not limited to, laminin; alpha1 (I)-CB3435438, rat; and laminin binding inhibitor.

Leptin peptides including, but not limited to, leptin 93-105, human; leptin 22-56, rat; Tyr-leptin 26-39, human; and leptin 116-130, amide, mouse.

Leucokinin peptides including, but not limited to, leucomyosuppressin (LMS); leucopyrokinin (LPK); leucokinin I; leucokinin II; leucokinin III; leucokinin IV; leucokinin VI; leucokinin VII; and leucokinin VIII.

Luteinizing hormone-releasing hormone peptides including, but not limited to, antide; Gn-RH II, chicken; luteinizing hormone-releasing hormone (LH-RH) (GnRH); biotinyl-LH-RH; cetrorelix (D-20761); [D-Ala⁶]-LH-RH; [Gln⁸]-LH-RH (Chicken LH-RH); [DLeu⁶, Val⁷] LH-RH 1-9, ethyl amide; [D-Lys⁶]-LH-RH; [D-Phe², Pro³, D-Phe⁶]-LH-RH; [DPhe², DAla⁶] LH-RH; [Des-Gly¹⁰]-LH-RH, ethyl amide; [D-Ala⁶, Des-Gly¹⁰]-LH-RH, ethyl amide; [DTrp⁶]-LH-RH, ethyl amide; [D-Trp⁶, Des-Gly¹⁰]-LH-RH, ethyl amide (Deslorelin); [DSer(But)⁶, Des-Gly¹⁰]-LH-RH, ethyl amide; ethyl amide; leuprolide; LH-RH 4-10; LH-RH 7-10; LH-RH, free acid; LH-RH, lamprey; LH-RH, salmon; [Lys⁸]-LH-RH; [Trp⁷,Leu⁸] LH-RH, free acid; and [(t-Bu)DSer⁶, (Aza)Gly¹⁰]-LH-RH.

Mastoparan peptides including, but not limited to, mastoparan; mas7; mas8; mas 17; and mastoparan X.

Mast cell degranulating peptides including, but not limited to, mast cell degranulating peptide HR-1; and mast cell degranulating peptide HR-2.

Melanocyte stimulating hormone (MSH) peptides including, but not limited to, [Ac-Cys⁴,DPhe⁷,Cys¹⁰] alpha-MSH 4-13, amide; alpha-melanocyte stimulating hormone; alpha-MSH, free acid; beta-MSH, porcine; biotinyl-alpha-melanocyte stimulating hormone; biotinyl-[Nle⁴, D-Phe¹] alpha-melanocyte stimulating hormone; [Des-Acetyl]-alpha-MSH; [DPhe¹]-alpha-MSH, amide; gamma-1-MSH, amide; [Lys⁰]-gamma-1-MSH, amide; MSH release inhibiting factor, amide; [Nle⁴]-alpha-MSH, amide; [Nle⁴, D-Phe⁷]-alpha-MSH; N-Acetyl, [Nle⁴,DPhe⁷] alpha-MSH 4-10, amide; beta-MSH, human; and gamna-MSH.

Morphiceptin peptides including, but not limited to, morphiceptin (beta-casomorphin 14 amide); [D-Pro⁴]-morphiceptin; and [N-MePhe³,D-Pro⁴]-morphiceptin.

Motilin peptides including, but not limited to, motilin, canine; motilin, porcine; biotinyl-motilin, porcine; and [Leu¹³]-motilin, porcine.

Neuro-peptides including, but not limited to, Ac-Asp-Glu; achatina cardio-excitatory peptide-1 (ACEP-1) (Achatina fulica); adipokinetic hormone (AKH) (Locust); adipokinetic hormone (Heliothis zea and Manduca sexta); alytesin; Tabanus atratus adipokinetic hormone (Taa-AKH); adipokinetic hormone II (Locusta migratoria); adipokinetic hormone II (Schistocera gregaria); adipokinetic hormone III (AKH-3); adipokinetic hormone G (AKH-G) (Gryllus bimaculatus); allatotropin (AT) (Manduca sexta); allatotropin 6-13 (Manduca sexta); APGW amide (Lymnaea stagnalis); buccalin; cerebellin; [Des-Ser¹]-cerebellin; corazonin (American Cockroach Periplaneta americana); crustacean cardioactive peptide (CCAP); crustacean erythrophore; DF2 (Procambarus clarkii); diazepam-binding inhibitor fragment, human; diazepam binding inhibitor fragment (ODN); eledoisin related peptide; FMRF amide (molluscan cardioexcitatory neuro-peptide); Gly-Pro-Glu (GPE), human; granuliberin R; head activator neuropeptide; [His⁷]-corazonin; stick insect hypertrehalosaemic factor II; Tabanus atratus hypotrehalosemic hormone (Taa-HoTH); isoguvacine hydrochloride; bicuculline methiodide; piperidine-4-sulphonic acid; joining peptide of proopiomelanocortin (POMC), bovine; joining peptide, rat; KSAYMRF amide (P. redivivus); kassinin; kinetensin; levitide; litorin; LUQ 81-91 (Aplysia californica); LUQ 83-91 (Aplysia californica); myoactive peptide I (Periplanetin CC-1) (Neuro-hormone D); myoactive peptide II (Periplanetin CC-2); myomodulin; neuron specific peptide; neuron specific enolase 404443, rat; neuropeptide FF; neuropeptide K, porcine; NEI (prepro-MCH 131-143) neuropeptide, rat; NGE (prepro-MCH 110-128) neuropeptide, rat; NFI (Procambarus clarkii); PBAN-1 (Bombyx mori); Hez-PBAN (Heliothis zea); SCPB (cardioactive peptide from aplysia); secretoneurin, rat; uperolein; urechistachykinin I; urechistachykinin II; xenopsin-related peptide 1; xenopsin-related peptide II; pedal peptide (Pep), aplysia; peptide Fl, lobster; phyllomedusin; polistes mastoparan; proctolin; ranatensin; Ro I (Lubber Grasshopper, Romalea microptera); Ro II (Lubber Grasshopper, Romalea microptera); SALMF amide 1 (SI); SALMF amide 2 (S2); and SCPA.

Neuropeptide Y (NPY) peptides including, but not limited to, [Leu³¹,Pro³⁴]-neuropeptide Y, human; neuropeptide F (Moniezia expansa); B1BP3226 NPY antagonist; Bis (31/31′) {[Cys³¹, Trp³², Nva³⁴] NPY 31-36}; neuropeptide Y, human, rat; neuropeptide Y 1-24 amide, human; biotinyl-neuropeptide Y; [D-Tyr^{27,36}, D-Thr³²]-NPY 27-36; Des 10-17 (cyclo 7-21) [Cys^{7,21}, Pro³⁴]-NPY; C2-NPY; [Leu³¹, Pro³⁴] neuropeptide Y, human; neuropeptide Y, free acid, human; neuropeptide Y, free acid, porcine; prepro NPY 68-97, human; N-acetyl-[Leu²⁸, Leu³¹] NPY 24-36; neuropeptide Y, porcine; [D-Trp³²]-neuropeptide Y, porcine; [D-Trp³²] NPY 1-36, human; [Leu¹⁷,DTrp³²] neuropeptide Y, human; [Leu³¹, Pro³⁴]-NPY, porcine; NPY 2-36, porcine; NPY 3-36, human; NPY 3-36, porcine; NPY 13-36, human; NPY 13-36, porcine; NPY 16-36. porcine; NPY 18-36, porcine; NPY 20-36; NFY 22-36; NPY 26-36; [Pro³⁴]-NPY 1-36, human; [Pro³⁴]-neuropeptide Y, porcine; PYX-1; PYX-2; T4-[NPY(33-36)]4; and Tyr(OMe)²¹]-neuropeptide Y, human.

Neurotropic factor peptides including, but not limited to, glial derived neurotropic factor (GDNF); brain derived neurotropic factor (BDNF); and ciliary neurotropic factor (CNTF).

Orexin peptides including, but not limited to, orexin A; orexin B, human; orexin B, rat, mouse.

Opioid peptides including, but not limited to, alpha-casein fragment 90-95; BAM-18P; casomokinin L; casoxin D; crystalline; DALDA; dermenkephalin (deltorphin) (Phylomedusa sauvagei); [D-Ala²]-deltorphin I; [D-Ala²]-deltorphin II; endomorphin-1; endomorphin-2; kyotorphin; [DArg²]-kyotorphin; morphin tolerance peptide; morphine modulating peptide, C-terminal fragment; morphine modulating neuropeptide (A-18-F-NH2); nociceptin [orphanin FQ] (ORLI agonist); TIPP; Tyr-MIF-1; Tyr-W-MIF-1; valorphin; LW-hemorphin-6, human; Leu-valorphin-Arg; and Z-Pro-D-Leu.

Oxytocin peptides including, but not limited to, [Asu⁶]-oxytocin; oxytocin; biotinyl-oxytocin; [Thr⁴, Gly⁷]-oxytocin; and tocinoic acid ([Ile³]-pressinoic acid).

PACAP (pituitary adenylating cyclase activating peptide) peptides including, but not limited to, PACAP 1-27, human, ovine, rat; PACAP (1-27)-Gly-Lys-Arg-NH2, human; [Des-Gln¹⁶]-PACAP 6-27, human, ovine, rat; PACAP38, frog; PACAP27-NH2, human, ovine, rat; biotinyl-PACAP27-NH2, human, ovine, rat; PACAP 6-27, human, ovine, rat; PACAP38, human, ovine, rat; biotinyl-PACAP38, human, ovine, rat; PACAP 6-38, human, ovine, rat; PACAP27-NH2, human, ovine, rat; biotinyl-PACAP27-NH2, human, ovine, rat; PACAP 6-27, human, ovine, rat; PACAP38, human, ovine, rat; biotinyl-PACAP38, human, ovine, rat; PACAP 6-38, human, ovine, rat; PACAP38 16-38, human, ovine, rat; PACAP38 31-38, human, ovine, rat; PACAP38 31-38, human, ovine, rat; PACAP-related peptide (PRP), human; and PACAP-related peptide (PRP), rat.

Pancreastatin peptides including, but not limited to, chromostatin, bovine; pancreastatin (hPST-52) (chromogranin A 250-301, amide); pancreastatin 24-52 (hPST-29), human; chromogranin A 286-301, amide, human; pancreastatin, porcine; biotinyl-pancreastatin, porcine; [Nle⁸]-pancreastatin, porcine; [Tyr⁰,Nle⁸]-pancreastatin, porcine; [Tyr⁰]-pancreastatin, porcine; parastatin 1-19 (chromogranin A 347-365), porcine; pancreastatin (chromogranin A 264-314-amide, rat; biotinyl-pancreastatin (biotinyl-chromogranin A 264-314-amide; [Tyr⁰]-pancreastatin, rat; pancreastatin 26-51, rat; and pancreastatin 33-49, porcine.

Pancreatic polypeptides including, but not limited to, pancreatic polypeptide, avian; pancreatic polypeptide, human; C-fragment pancreatic polypeptide acid, human; C-fragment pancreatic polypeptide amide, human; pancreatic polypeptide (Rana temporaria); pancreatic polypeptide, rat; and pancreatic polypeptide, salmon.

Parathyroid hormone peptides including, but not limited to, [Asp⁷⁶]-parathyroid hormone 39-84, human; [Asp⁷⁶]-parathyroid hormone 53-84, human; [Asn⁷⁶]-parathyroid hormone 1-84, hormone; [Asn⁷⁶]-parathyroid hormone 64-84, human; [Asn⁸, Leu¹⁸]-parathyroid hormone 1-34, human; [Cys^{5,28}]-parathyroid hormone 1-34, human; hypercalcemia malignancy factor 140; [Leu¹⁸]-parathyroid hormone 1-34, human; [(Lys(biotinyl)¹³, Nle^{8,18}, Tyr³⁴]-parathyroid hormone 1-34 amide; [Nle^{8,18}, Tyr³⁴]-parathyroid hormone 1-34 amide; [Nle^{8,18}, Tyr³⁴]-parathyroid hormone 3-34 amide, bovine; [Nle^{8,18}, Tyr³⁴]-parathyroid hormone 1-34, human; [Nle^{8,8}, Tyr³⁴]-parathyroid hormone 1-34 amide, human; [Nle^{8,18}, Tyr³⁴]-parathyroid hormone 3-34 amide, human; [Nle^{8,18}, Tyr³⁴]-parathyroid hormone 7-34 amide, bovine; [Nle^{8,18}, Tyr³⁴]-parathyroid hormone 1-34 amide, rat; parathyroid hormone 44-68, human; parathyroid hormone 1-34, bovine; parathyroid hormone 3-34, bovine; parathyroid hormone 1-31 amide, human; parathyroid hormone 1-34, human; parathyroid hormone 13-34, human; parathyroid hormone 1-34, rat; parathyroid hormone 1-38, human; parathyroid hormone 1-44, human; parathyroid hormone 2848, human; parathyroid hormone 39-68, human; parathyroid hormone 39-84, human; parathyroid hormone 53-84, human; parathyroid hormone 69-84, human; parathyroid hormone 70-84, human; [Pro³⁴]-peptide YY (PYY), human; [Tyr⁰]-hypercalcemia malignancy factor 1-40; [Tyr¹-parathyroid hormone 1-44, human; [Tyr⁰]-parathyroid hormone 1-34, human; [Tyr¹]-parathyroid hormone 1-34, human; [Tyr⁷]-parathyroid hormone 27-48, human; [Tyr³⁴]-parathyroid hormone 7-34 amide, bovine; [Tyr⁴³]-parathyroid hormone 43-68, human; [Tyr⁵², Asn⁷⁶]-parathyroid hormone 52-84, human; and [Tyr⁶³]-parathyroid hormone 63-84, human.

Parathyroid hormone (PTH)-related peptides including, but not limited to, PTHRP ([Tyr³⁶]-PTHrP 1-36 amide), chicken; hHCF-(1-34)-NH2 (humoral hypercalcemic factor), human; PTH-related protein 1-34, human; biotinyl-PTH-related protein 1-34, human; [Tyr⁰]-PTH-related protein 1-34, human; [Tyr³⁴]-PTH-related protein 1-34 amide, human; PTH-related protein 1-37, human; PTH-related protein 7-34 amide, human; PTH-related protein 38-64 amide, human; PTH-related protein 67-86 amide, human; PTH-related protein 107-111, human, rat, mouse; PTH-related protein 107-111 free acid; PTH-related protein 107-138, human; and PTH-related protein 109-111, human.

Peptide T peptides including, but not limited to, peptide T; [D-Ala¹]-peptide T; and [D-Ala¹]-peptide T amide.

Prolactin-releasing peptides including, but not limited to, prolactin-releasing peptide 31, human; prolactin-releasing peptide 20, human; prolactin-releasing peptide 31, rat; prolactin-releasing peptide 20, rat; prolactin-releasing peptide 31, bovine; and prolactin-releasing peptide 20, bovine.

Peptide YY (PYY) peptides including, but not limited to, PYY, human; PYY 3-36, human; biotinyl-PYY, human; PYY, porcine, rat; and [Leu³¹, Pro³⁴]-PYY, human.

Renin substrate peptides including, but not limited to, acetyl, angiotensinogen 1-14, human; angiotensinogen 1-14, porcine; renin substrate tetradecapeptide, rat; [Cys⁸]-renin substrate tetradecapeptide, rat; [(Leu⁸]-renin substrate tetradecapeptide, rat; and [Val⁸]-renin substrate tetradecapeptide, rat.

Secretin peptides including, but not limited to, secretin, canine; secretin, chicken; secretin, human; biotinyl-secretin, human; secretin, porcine; and secretin, rat.

Somatostatin (GIF) peptides including, but not limited to, BIM-23027; biotinyl-somatostatin; biotinylated cortistatin 17, human; cortistatin 14, rat; cortistatin 17, human; [Tyr]-cortistatin 17, human; cortistatin 29, rat; [D-Trp⁸]-somatostatin; [Trp⁸,DCys ⁴]-somatostatin; [DTrp⁸,Tyr¹¹]-somatostatin; [D-Trp¹¹]-somatostatin; NTB (Naltriben); [Nle⁸]-somatostatin 1-28; octreotide (SMS 201-995); prosomatostatin 1-32, porcine; [Tyr⁰]-somatostatin; [Tyr¹]-somatostatin; [Tyr¹]-somatostatin 28 (1-14); [Tyr¹¹]-somatostatin; [Tyr⁰, D-Trp⁸]-somatostatin; somatostatin; somatostatin antagonist; somatostatin-25; somatostatin-28; somatostatin 28 (1-12); biotinyl-somatostatin-28; [Tyr¹]-somatostatin-28; [Leu⁸, D-Trp²², Tyr¹⁵]-somatostatin-28; biotinyl-[Leu⁸, D-Trp²², Tyr²⁵]-somatostatin-28; somatostatin-28 (1-14); and somatostatin analog, RC-160.

Substance P peptides including, but not limited to, G protein antagonist-2; Ac-[Arg⁶, Sar⁹, Met(02)¹¹]-substance P 6-11; [Arg³]-substance P; Ac-Trp-3,5-bis(trifluoromethyl) benzyl ester; Ac-[Arg⁶, Sar⁹, Met(02)¹¹]-substance P 6-11; [D-Ala⁴]-substance P 4-11; [Tyr⁶, D-Phe⁷, D-His⁹]-substance P 6-11 (sendide); biotinyl-substance P; biotinyl-NTE[Arg³]-substance P; [Tyr⁸]-substance P; [Sar⁹, Met(02)¹¹]-substance P; [D-Pro², D-Trp^{7,9}]-substance P; [D-Pro⁴, O-Trp ^{7,9}]-substance P 4-11; substance P 4-11; [DTrp^{2,7,9}]-substance P; [(Dehydro)Pro^{2,4}, Pro⁹]-substance P; [Dehydro-Pro⁴]-substance P 4-11; [Glp⁵,(Me)Phe⁸,Sar⁹]-substance P 5-11; [Glp⁵,Sar⁹]-substance P 5-11; [Glp⁵]-substance P 5-11; hepta-substance P (substance P 5-11); hexa-substance P(substance P 6-11); [MePhe⁸,Sar⁹]-substance P; [Nle¹¹]-substance P; Octa-substance P(substance P 4-11); [pGlu¹]-hexa-substance P ([pGlu⁶]-substance P 6-11); [pGlu⁶, D-Pro⁹]-substance P 6-11; [(pNO2)Phe⁷Nle¹¹]-substance P; penta-substance P (substance P 7-11); [Pro⁹]-substance P; GR73632, substance P 7-11; [Sar⁴]-substance P 4-11; [Sar⁹]-substance P; septide ([pGlu⁶, Pro⁹]-substance P 6-11); spantide I; spantide II; substance P; substance P, cod; substance P, trout; substance P antagonist; substance P-Gly-Lys-Arg; substance P 14; substance P 1-6; substance P 1-7; substance P 1-9; deca-substance P (substance P 2-11); nona-substance P (substance P 3-11); substance P tetrapeptide (substance P 8-11); substance P tripeptide (substance P 9-11); substance P, free acid; substance P methyl ester; and [Tyr⁸,Nle¹¹] substance P.

Tachykinin peptides including, but not limited to, [Ala⁵, beta-Ala⁸] neurokinin A 4-10; eledoisin; locustatachykinin I (Lom-TK-1) (Locusta migratoria); locustatachykinin II (Lom-TK-II) (Locusta migratoria); neurokinin A 4-10; neurokinin A (neuromedin L, substance K); neurokinin A, cod and trout; biotinyl-neurokinin A (biotinyl-neuromedin L, biotinyl-substance K); [Tyr⁰]-neurokinin A; [Tyr⁶]-substance K; FR64349; [Lys3, Glys-(R)-gamma-lactam-Leu⁹]-neurokinin A 3-10; GR83074; GR87389; GR94800; [Beta-Ala⁸]-neurokinin A 4-10; [Nle¹⁰]-neurokinin A 4-10; [Trp⁷, beta-Ala⁸]-neurokinin A 4-10; neurokinin B (neuromedin K); biotinyl-neurokinin B (biotinyl-neuromedin K); [MePhe⁷]-neurokinin B; [Pro⁷]-neurokinin B; [Tyr⁰]-neurokinin B; neuromedin B, porcine; biotinyl-neuromedin B, porcine; neuromedin B-30, porcine; neuromedin B-32, porcine; neuromedin B receptor antagonist; neuromedin C, porcine; neuromedin N, porcine; neuromedin (U-8), porcine; neuromedin (U-25), porcine; neuromedin U, rat; neuropeptide-gamma (gamma-preprotachykinin 72-92); PG-KII; phyllolitorin; [Leu⁸]-phyllolitorin (Phyllomedusa sauvagei); physalaemin; physalaemin 1-11; scyliorhinin II, amide, dogfish; senktide, selective neurokinin B receptor peptide; [Ser2]-neuromedin C; beta-preprotachykinin 69-91, human; beta-preprotachykinin 111-129, human; tachyplesin I; xenopsin; and xenopsin 25 (xenin 25), human.

Thyrotropin-releasing hormone (TRH) peptides including, but not limited to, biotinyl-thyrotropin-releasing hormone; [Glu¹]-TRH; His-Pro-diketopiperazine; [3-Me-His²]-TRH; pGlu-Gln-Pro-amide; pGlu-His; [Phe²]-TRH; prepro TRH 53-74; prepro TRH 83-106; prepro-TRH 160-169 (Ps4, TRH-potentiating peptide); prepro-TRH 178-199; thyrotropin-releasing hormone (TRH); TRH, free acid; TRH-SH Pro; and TRH precursor peptide.

Toxin peptides including, but not limited to, omega-agatoxin TK; agelenin, (spider, Agelena opulenta); apamin (honeybee, Apis mellifera); calcicudine (CaC) (green mamba, Dedroaspis angusticeps); calciseptine (black mamba, Dendroaspis polylepis polylepis); charybdotoxin (ChTX) (scorpion, Leiurus quinquestriatus var. hebraeus); chlorotoxin; conotoxin GI (marine snail, Conus geographus); conotoxin GS (marine snail, Conus geographus); conotoxin MI (Marine Conus magus); alpha-conotoxin EI, Conus ermineus; alpha-conotoxin SIA; alpha-conotoxin 1 ml; alpha-conotoxin SI (cone snail, Conus striatus); micro-conotoxin GIIIB (marine snail, Conus geographus); omega-conotoxin GVIA (marine snail, Conus geographus); omega-conotoxin MVIIA (Conus magus); omega-conotoxin MVIIC (Conus magus); omega-conotoxin SVIB (cone snail, Conus striatus); endotoxin inhibitor; geographutoxin I (GTX-I) (μ-Conotoxin GIIIA); iberiotoxin (IbTX) (scorpion, Buthus tamulus); kaliotoxin 1-37; kaliotoxin (scorpion, Androct-onus mauretanicus mauretanicus); mast cell-degranulating peptide (MCD-peptide, peptide 401); margatoxin (MgTX) (scorpion, Centruriodes Margaritatus); neurotoxin NSTX-3 (pupua new guinean spider, Nephilia maculata); PLTX-II (spider, Plectreurys tristes); scyllatoxin (leiurotoxin I); and stichodactyla toxin (ShK).

Vasoactive intestinal peptides (VIP/PHI) including, but not limited to, VIP, human, porcine, rat, ovine; VIP-Gly-Lys-Arg-NH2; biotinyl-PHI (biotinyl-PHI-27), porcine; [Glp¹⁶] VIP 16-28, porcine; PHI (PHI-27), porcine; PHI (PHI-27), rat; PHM-27 (PHI), human; prepro VIP 81-122, human; prepro VIP/PHM 111-122; prepro VIP/PHM 156-170; biotinyl-PHM-27 (biotinyl-PHI), human; vasoactive intestinal contractor (endothelin-beta); vasoactive intestinal octacosa-peptide, chicken; vasoactive intestinal peptide, guinea pig; biotinyl-VIP, human, porcine, rat; vasoactive intestinal peptide 1-12, human, porcine, rat; vasoactive intestinal peptide 10-28, human, porcine, rat; vasoactive intestinal peptide 11-28, human, porcine, rat, ovine; vasoactive intestinal peptide (cod, Gadus morhua); vasoactive intestinal peptide 6-28; vasoactive intestinal peptide antagonist; vasoactive intestinal peptide antagonist ([Ac-Tyr¹, D-Phe²]-GHRF 1-29 amide); vasoactive intestinal peptide receptor antagonist (4-Cl-D-Phe⁶, Leu¹⁷]-VIP); and vasoactive intestinal peptide receptor binding inhibitor, L-8-K.

Vasopressin (ADH) peptides including, but not limited to, vasopressin; [Asu¹⁶,Arg⁸]-vasopressin; vasotocin; [Asu¹⁶,Arg⁸]-vasotocin; [Lys⁸]-vasopressin; pressinoic acid; [Arg⁸]-desamino vasopressin desglycinamide; [Arg⁸]-vasopressin (AVP); [Arg⁸]-vasopressin desglycinamide; biotinyl-[Arg⁸]-vasopressin (biotinyl-AVP); [D-Arg⁸]-vasopressin; desamino-[Arg⁸]-vasopressin; desamino-[D-Arg⁸]-vasopressin (DDAVP); [deamino-[D-3-(3′-pyridyl-Ala)]-[Arg⁸]-vasopressin; [1-(beta-Mercapto-beta, beta-cyclopentamethylene propionic-acid), 2-(O-methyl)tyrosine]-[Arg⁸]-vasopressin; vasopressin metabolite neuropeptide [pGlu⁴, Cys⁶]; vasopressin metabolite neuropeptide [pGlu⁴, Cys⁶]; [Lys⁸]-deamino vasopressin desglycinamide; [Lys⁸]-vasopressin; [Mpr¹,Val⁴,DArg⁸]-vasopressin; [Phe², Ile³, Orn⁸]-vasopressin ([Phe², Orn⁸]-vasotocin); [Arg⁸]-vasotocin; and [d(CH2)₅, Tyr(Me)², rn8]-vasotocin.

Virus related peptides including, but not limited to, fluorogenic human CMV protease substrate; HCV core protein 59-68; HCV NS4A protein 1840 (JT strain); HCV NS4A protein 21-34 (JT strain); hepatitis B virus receptor binding fragment; hepatitus B virus pre-S region 120-145; [Ala¹²⁷]-hepatitus B virus pre-S region 120-131; herpes virus inhibitor 2; HIV envelope protein fragment 254-274; HIV gag fragment 129-135; HIV substrate; P 18 peptide; peptide T; [3,5 diiodo-Tyr⁷] peptide T; R1SK HIV-1 inhibitory peptide; T20; T21; V3 decapeptide P 18-110; and virus replication inhibiting peptide.

While certain analogs, fragments, and/or analog fragments of the various polypeptides have been described above, it is to be understood that other analogs, fragments, and/or analog fragments that retain all or some of the activity of the particular polypeptide may also be useful in embodiments of the present invention. Analogs may be obtained by various means, as will be understood by those skilled in the art. For example, certain amino acids may be substituted for other amino acids in a polypeptide without appreciable loss of interactive binding capacity with structures such as, for example, antigen-binding regions of antibodies or binding sites on substrate molecules. As the interactive capacity and nature of a polypeptide drug defines its biological functional activity, certain amino acid sequence substitutions can be made in the amino acid sequence and nevertheless remain a polypeptide with like properties.

5.2.1.3 Thiol-Containing Drugs

Thiol-containing drugs can be modified to be in the form of the prodrugs described herein. For example, the thiol group can be reacted to form a thiocarbamate moiety (i.e., —SC(O)NHR′), which includes an active NH group which can be further reacted to form an additional carbamate moiety.

Thiol-containing drugs include, for example, thiol-containing peptides and proteins. There are several known thiol-containing anti-inflammatory drugs, thiol-containing antirheumatic drugs, thiol-containing peptidomimetic inhibitors, thiol-containing angiotensin converting enzyme inhibitors, cysteine proteases, and thiol-containing antimicrobial heterocycles.

Examples include glutathione (a coenzyme that functions in several redox reactions in the body), metallothiones, homo-cysteine, N-acetyl cysteine (NAC), D-penicillamine, captopril (a thiol-containing ACE inhibitor), 6-mercaptopurine, mercaprol, dimercaptopropanesulfonate, the cysteine protease cathepsin B, cathepsin K, L, and S, thioredoxin reductase, and transcription factors such as NFk-B and its regulator IKK.

5.2.1.4 Carboxylic Acid-Containing Drugs

Carboxylic acid-containing drugs can be modified to be in the form of the prodrugs described herein. For example, the carboxylic acid group can be reacted to form an amide moiety (—C(O)NHR′), thioamide moiety (i.e., —C(S)NHR′), imide moiety (—C(O)—NHC(O)R′), thioimide moiety (—C(S)—NHC(S)R′), and the like, each of which includes an active NH group which can be further reacted to form an additional carbamate moiety.

5.2.1.5 Phosphoramide-Containing Drugs

Phosphoramide-containing drugs can be modified to be in the form of the prodrugs described herein. For example, the phosphoramide group (—P(O)₂NHR′) includes an active NH group which can be further reacted to form a carbamate moiety. An example of a phosphoramide-containing drug is N,N′,N″-triethylenethiophosphoramide (THIOTEPA), an anti-tumor agent.

5.2.2 Modifying Moiety (R)

The “modifying moieties” provide the prodrug with certain desired properties. For example, the moiety can modify the drug by providing the drug with improved stability in certain environments, increasing the drug's hydrophilicity or hydrophobicity, increasing the drug's ability to cross the cell membrane, increasing the drug's ability to cross the blood-brain barrier, or targeting the drug to a certain receptor, cell (for example, a tumor cell), tissue, or organ.

5.2.3 Moieties that Effect Stability, Solubility, and/or Biological Activity

There are numerous moieties that can be attached to the biologically active agents to form the prodrugs described herein that modify the stability, solubility, and/or biological activity of the drug. Examples include hydrophilic polymers or oligomers (i.e., molecules with between 2 and 100 repeating units), amphiphilic polymers or oligomers, and lipophilic polymers or oligomers.

The polymers (or shorter chain oligomers) can include weak or degradable linkages in their backbones. For example, the polyalkylene glycols can include hydrolytically unstable linkages, such as lactide, glycolide, carbonate, ester, carbamate and the like, which are susceptible to hydrolysis.

This allows the polymers to be cleaved into lower molecular weight fragments. Examples of such polymers are described, for example, in U.S. Pat. No. 6,153,211 to Hubbell et al.

Representative hydrophilic, amphiphilic, and lipophilic polymers and oligomers are described in more detail below.

5.2.3.1 Hydrophilic Polymers and Oligomers

The hydrophilic moiety may be various hydrophilic moieties as will be understood by those skilled in the art including, but not limited to, polyalkylene glycol moieties, other hydrophilic polymers, sugar moieties, polysorbate moieties, and combinations thereof.

5.2.3.2 Polyalkylene Glycol Moieties

Poly(alkylene glycols) are compounds with repeat alkylene glycol units. In some embodiments, the units are all identical (i.e., poly(ethylene glycol) or poly(propylene glycol). In other embodiments, the alkylene units are different (i.e., poly(ethylene-co-propylene glycol, or PLURONICS®). The polymers can be random copolymers (for example, where ethylene oxide and propylene oxide are co-polymerized) or branched or graft copolymers.

Poly(ethylene glycol), or PEG, is a preferred polyalkylene glycol, and is useful in biological applications because it has highly desirable properties and is generally regarded as safe (GRAS) by the Food and Drug Administration. PEG has the formula —(CH₂CH₂O)ₙ, where n can range from about 2 to about 4000 or more. PEG typically is colorless, odorless, water-soluble or water-miscible (depending on molecular weight), heat stable, chemically inert, hydrolytically stable, and generally nontoxic. Poly(ethylene glycol) is also biocompatible, and typically does not produce an immune response in the body. When attached (directly or indirectly) to a biologically active agent, it masks the agent and can reduce any immune response so that an organism can tolerate the presence of the agent. Those prodrugs including a PEG moiety are typically substantially non-toxic and do not tend to produce substantial immune responses, or cause clotting or other undesirable effects.

In some embodiments, it is preferred to have a mono-dispersed polyalkylene glycol, rather than a polydispersed polyalkylene glycol. By mono-disperse, it is meant that the polyalkylene glycol can have a single molecular weight, or a relatively narrow range of molecular weights. Generally, mono-dispersed polyalkylene oxides have relatively fewer subunits than polydispersed polyalkylene oxides. The monodispersed polyalkylene glycol may have as few as 1, 2, 3, polyalkylene glycol subunits, and in some embodiments, the monodispersed polyalkylene glycol has between about 1 and 200 subunits, for example, between about 1 and 50 subunits, or between about 5 and 10 subunits. In some embodiments, the polyalkylene glycol has between a lower limit of 1, 2, 3, 4, 5, 6, 7, 8, 9, 10, 11, 12, 13, 14, 15, 16, 17, 18, or 19 polyalkylene glycol subunits and an upper limit of 2, 3, 4, 5, 6, 7, 8, 9, 10, 11, 12, 13, 14, 15, 16, 17, 18, 19, or 20 polyalkylene glycol subunits. In some embodiments, a polyalkylene glycol having between about 1 and 50 subunits is preferred for preparing the prodrugs described herein.

One advantage of using the relatively low molecular weight, monodispersed polymers is that they form easily defined prodrug molecules, which can facilitate both reproducible synthesis and FDA approval.

In other embodiments, the PEG is polydispersed, and has a molecular weight of from about 200 to about 100,000 Da. In this embodiment, the PEG can be a linear polymer with a hydroxyl group at each terminus (before being conjugated to the remainder of the prodrug). The PEG can also be an alkoxy PEG, such as methoxy-PEG (or mPEG), where one terminus is a relatively inert alkoxy group, while the other terminus is a hydroxyl group (that is coupled to the remainder of the prodrug). The PEG can also be branched, which can in one embodiment be represented as R(—PEG-OH)ₘ in which R represents a central (typically polyhydric) core agent such as pentaerythritol or glycerol, and m represents the number of arms. Each branch can be different and can be terminated, for example, with ethers and/or esters. The number of arms m can range from three to a hundred or more, and one or more of the terminal hydroxyl groups can be coupled to the remainder of the prodrug, or otherwise subject to chemical modification. Other branched PEG include those represented by the formula (CH₃O-PEG-)ₚ R-Z, where p equals 2 or 3, R represents a central core such as lysine or glycerol, and Z represents a group such as carboxyl that is subject to ready chemical activation. Still another branched form, the pendant PEG, has reactive groups, such as carboxyls, along the PEG backbone rather than, or in addition to, the end of the PEG chains. Forked PEG can be represented by the formula PEG(-LCHX₂)ₙ is another form of branched PEG, where L is a linking group and X is an activated terminal group. The term poly(ethylene glycol) or PEG represents or includes all of the above forms.

5.2.3.3 Other Hydrophilic Polymers and Oligomers

Other hydrophilic polymers can also be used. Examples include poly(oxyethylated polyols) such as poly(oxyethylated glycerol), poly(oxyethylated sorbitol), and poly(oxyethylated glucose); poly(vinyl alcohol) (“PVA”); dextran; carbohydrate-based polymers and the like. The polymers can be homopolymers or random or block copolymers and terpolymers based on the monomers of the above polymers, straight chain or branched.

Specific examples of suitable additional polymers include, but are not limited to, poly(oxazoline), difunctional poly(acryloylmorpholine) (“PAcM”), and poly(vinylpyrrolidone)(“PVP”). PVP and poly(oxazoline) are well known polymers in the art and their preparation should be readily apparent to the skilled artisan. PAcM and its synthesis and use are described in U.S. Pat. Nos. 5,629,384 and 5,631,322, the contents of which are incorporated herein by reference in their entirety.

5.2.3.4 Sugar Moieties

The prodrugs described herein can include sugar moieties, as such as known by those skilled in the art. In general, the sugar moiety is a carbohydrate product of at least one saccharose group. Representative sugar moieties include, but are not limited to, glycerol moieties, mono-, di-, tri-, and oligosaccharides, and polysaccharides such as starches, glycogen, cellulose and polysaccharide gums. Specific monosaccharides include C₆ and above (preferably C₆ to C₈) sugars such as glucose, fructose, mannose, galactose, ribose, and sedoheptulose; di- and trisaccharides include moieties having two or three monosaccharide units (preferably C5 to C8) such as sucrose, cellobiose, maltose, lactose, and raffinose.

5.2.3.5 Polysorbate Polymers and Oligomers

The polysorbate moiety may be various polysorbate moieties as will be understood by those skilled in the art including, but are not limited to, sorbitan esters, and polysorbate derivatized with polyoxyethylene.

5.2.4 Biocompatible Water-Soluble Polycationic Polymers and Oligomers

In some embodiments, biocompatible water-soluble polycationic polymers can be used. Biocompatible water-soluble polycationic polymers include, for example, any polymer having protonated heterocycles attached as pendant groups. “Water soluble” means that the entire polymer is soluble in aqueous solutions, such as buffered saline or buffered saline with small amounts of added organic solvents as cosolvents, at a temperature between 20 and 37° C. In some embodiments, the polymer itself is not sufficiently soluble in aqueous solutions per se but is brought into solution by grafting with water-soluble polymers such as polyethylene glycol chains. Examples include polyamines having amine groups on either the polymer backbone or the polymer sidechains, such as poly-L-lysine and other positively charged polyamino acids of natural or synthetic amino acids or mixtures of amino acids, including poly(D-lysine), poly(ornithine), poly(arginine), and poly(histidine), and nonpeptide polyamines such as poly(aminostyrene), poly(aminoacrylate), poly (N-methyl aminoacrylate), poly (N-ethylaminoacrylate), poly(N,N-dimethyl aminoacrylate), poly(N,N-diethylaminoacrylate), poly(aminomethacrylate), poly(N-methyl amino-methacrylate), poly(N-ethyl aminomethacrylate), poly(N,Ndimethyl aminomethacrylate), poly(N,N-diethyl aminomethacrylate), poly(ethyleneimine), polymers of quaternary amines, such as poly(N,N,N-trimethylaminoacrylate chloride), poly(methyacrylamidopropyltrimethyl ammonium chloride), and natural or synthetic polysaccharides such as chitosan.

5.2.4.1 Bioadhesive Polyanionic Polymers and Oligomers

Certain hydrophilic polymers appear to have potentially useful bioadhesive properties. Examples of such polymers are found, for example, in U.S. Pat. No. 6,197,346 to Mathiowitz, et al. Those polymers containing carboxylic groups (e.g., poly(acrylic acid)) exhibit bioadhesive properties, and also are easily conjugated with the remainder of the prodrug moiety described herein. Rapidly bioerodible polymers that expose carboxylic acid groups on degradation, such as poly(lactide-co-glycolide), polyanhydrides, and polyorthoesters, are also bioadhesive polymers. These polymers can be used to deliver the prodrugs to the gastrointestinal tract. As the polymers degrade, they can expose carboxylic acid groups to enable them to adhere strongly to the gastrointestinal tract, and can aid in the release of active drugs from the prodrugs described herein.

In some embodiments, R comprises a lipophilic moiety. The lipophilic moiety may be various lipophilic moieties as will be understood by those skilled in the art including, but not limited to, alkyl moieties, alkenyl moieties, alkynyl moieties, aryl moieties, arylalkyl moieties, alkylaryl moieties, fatty acid moieties, adamantantyl, and cholesteryl, as well as lipophilic polymers and/or oligomers.

The alkyl moiety can be a saturated or unsaturated, linear, branched, or cyclic hydrocarbon chain. In some embodiments, the alkyl moiety has at least 1, 2, 3, or more carbon atoms. In other embodiments, the alkyl moiety is a linear, saturated or unsaturated alkyl moiety having between a lower limit of 1, 2, 3, 4, 5, 6, 7, 8, 9, 10, 11, 12, 13, 14, 15, 16, 17, 18, or 19 carbon atoms and an upper limit of 2, 3, 4, 5, 6, 7, 8, 9, 10, 11, 12, 13, 14, 15, 16, 17, 18, 19, or 20 carbon atoms including for example, saturated, linear alkyl moieties such as methyl, ethyl, propyl, butyl, pentyl, hexyl, heptyl, octyl, nonyl, decyl, undecyl, dodecyl, tridecyl, tetradecyl, pentadecyl, hexadecyl, octadecyl, nonadecyl and eicosyl; saturated, branched alkyl moieties such as isopropyl, sec-butyl, tert-butyl, 2-methylbutyl, tert-pentyl, 2-methyl-pentyl, 3-methylpentyl, 2-ethylhexyl, 2-propylpentyl; and unsaturated alkyl moieties derived from the above saturated alkyl moieties including, but not limited to, vinyl, allyl, 1-butenyl, 2-butenyl, ethynyl, 1-propynyl, and 2-propynyl. In other embodiments, the alkyl moiety is a lower alkyl moiety. In still other embodiments, the alkyl moiety is a C₁ to C₃ lower alkyl moiety.

The alkyl groups can either be unsubstituted or substituted with one or more substituents, such as those described above with respect to R′, and such substituents preferably either do not interfere with the methods of synthesis of the prodrugs or with the biological activity of the prodrugs. Potentially interfering functionality can be suitably blocked with a protecting group so as to render the functionality non-interfering. Each substituent may be optionally substituted with additional non-interfering substituents. The term “non-interfering” characterizes the substituents as not adversely affecting any reactions to be performed in accordance with the process of this invention.

The fatty acid moiety may be various fatty acid moieties including natural or synthetic, saturated or unsaturated, linear or branched fatty acid moieties. In some embodiments, the fatty acid moiety has at least 2, 3, 4, or more carbon atoms. In other embodiments, the fatty acid moiety has between a lower limit of 2, 3, 4, 5, 6, 7, 8, 9, 10, 11, 12, 13, 14, 15, 16, 17, 18, 19, 20, 21, 22, or 23 carbon atoms and an upper limit of 3, 4, 5, 6, 7, 8, 9, 10, 11, 12, 13, 14, 15, 16, 17, 18, 19, 20, 21, 22, 23, or 24 carbon atoms.

When R is an aryl ring, the ring can be functionalized with a nucleophilic functional group (such as OH, SH, or NHR′) that is positioned so that it can react in an intramolecular fashion with the carbamate moiety and assist in its hydrolysis. In some embodiments, the nucleophilic group is protected with a protecting group capable of being hydrolyzed or otherwise degraded in vivo, with the result being that when the protecting group is deprotected, hydrolysis of the prodrug, and resultant release of the native drug, is facilitated. In some embodiments, R is not an aryl ring.

5.2.4.2 Amphiphilic Polymers and Oligomers

In some embodiments, R includes an amphiphilic moiety. Many polymers and oligomers are amphiphilic. These are often block co-polymers, branched copolymers or graft co-polymers that include hydrophilic and lipophilic moieties, which can be in the form of oligomers and/or polymers, such as straight chain, branched, or graft polymers or co-polymers.

The hydrophilic polymers or oligomers described above typically include at least one reactive functional group, for example, halo, hydroxyl, amine, thiol, sulfonic acid, carboxylic acid, isocyanate, epoxy, ester, and the like, which are often at the terminal end of the polymer. These reactive functional groups can be used to attach a lipophilic linear or branched chain alkyl, alkenyl, alkynyl, arylalkyl, or alkylaryl group, or a lipophilic polymer or oligomer, thereby increasing the lipophilicity of the hydrophilic polymers or oligomers (and thereby rendering them amphiphilic).

The lipophilic groups can, for example, be derived from mono- or di-carboxylic acids, or where appropriate, reactive equivalents of carboxylic acids such as anhydrides or acid chlorides. Examples of suitable precursors for the lipophilic groups are acetic acid, propionic acid, butyric acid, valeric acid, isobutyric acid, trimethylacetic acid, caproic acid, caprylic acid, heptanoic acid, capric acid, pelargonic acid, lauric acid, myristic acid, palmitic acid, stearic acid, behenic acid, lignoceric acid, ceratic acid, montanoic acid, isostearic acid, isononanoic acid, 2-ethylhexanoic acid, oleic acid, ricinoleic acid, linoleic acid, linolenic acid, erucic acid, soybean fatty acid, linseed fatty acid, dehydrated castor fatty acid, tall oil fatty acid, tung oil fatty acid, sunflower fatty acid, safflower fatty acid, acrylic acid, methacrylic acid, maleic anhydride, orthophthalic anhydride, terephthalic acid, isophthalic acid, adipic acid, azelaic acid, sebacic acid, tetrahydrophthalic anhydride, hexahydrophthalic anhydride, succinic acid and polyolefin carboxylic acids.

The terminal lipophilic groups need not be equivalent, i.e., the resulting copolymers can include terminal lipophilic groups that are the same or different. The lipophilic groups can be derived from more than one mono or di-functional alkyl, alkenyl, alkynyl, cycloalkyl, arylalkyl or alkylaryl group as defined above.

5.2.4.3 Salt-Forming Moieties

In some embodiments, R comprises a salt-forming moiety. The salt-forming moiety may be various suitable salt-forming moieties as will be understood by those skilled in the art including, but not limited to, carboxylate and ammonium. In some embodiments wherein R includes a salt forming moiety, the prodrug is provided in salt form. In these embodiments, the prodrug is associated with a suitable pharmaceutically acceptable counterion as will be understood by those skilled in the art including, but not limited to, negative ions such as chloro, bromo, iodo, phosphate, acetate, carbonate, sulfate, tosylate, and mesylate, or positive ions such as sodium, potassium, calcium, lithium, and ammonium.

R can include any hydrophilic moieties, lipophilic moieties, amphiphilic moieties, salt-forming moieties, and combinations thereof. In preferred embodiments, R is selected from the group consisting of (CH₂CH₂O)ₚCH₃ where p is an integer from 0 to 9; (CH₂)_{q}CH₃ where q is an integer from 1 to 9; CH₂CH₂(OCH₂CH₂)ᵣOH where r is an integer from 0 to 9; C(CH₂OH)₃; CH(CH₂OH)₂; C(CH₃)₃; CH(CH₃)₂; CH₂CH₂(OCH₂CH₂)ₙC(O)(CH₂),CH₃ where s is an integer from 0 to 9 and t is an integer from 1 to 9; and (CH₂CH₂O)_{y}C(O)(CH₂)ₙCH₃ where y is an integer from 0 to 9 and z is an integer from 1 to 9.

5.2.4.4 Targeting Moieties

The moiety R can be used to target the prodrug to a desired location, for example, a desired cell, tissue, or organ within the body. This can be done, for example, using antibodies, ligands for various cell surface receptors, and the like. Examples of suitable antibodies include, for example, chimeric, human, and humanized antibodies that bind to tumor antigens. Such antibodies are described, for example, in U.S. Pat. No. 6,512,097 to Marks et al.

In one embodiment, R is a receptor ligand recognized by a particular cell type or biological target, thereby targeting the prodrug drug. Where R includes a receptor ligand (e.g., an antibody or antigen fragment such as Fab or Fab2′; or RGD or an RGD-mimic) recognized by a particular cell type, covalently attached to it, the invention also features a method of targeting the prodrug drug to a particular cell type.

In another embodiment, the various R groups described above as hydrophilic, amphiphilic or lipophilic polymers or oligomers are end-capped or otherwise functionalized with folic acid or other epitopes, small ligands, chemomimetic analogs, antibody fragments, antibodies, arginine-glycine-aspartic acid (RGD), analogs for targeting integrins, or other targeting moieties.

Increased drug delivery across the blood-brain barrier can be achieved by conjugating moieties that impart increased transport or permeability to the parent drug. Generally, covalent polar lipid conjugates can be prepared to facilitate transport across the blood-brain barrier. See, e.g., Duncan, “Drug-polymer conjugates: potential for improved chemotherapy,” Anticancer Drugs 3: 175-210 (1992); Senter et al., “Activation of Prodrugs by Antibody-Enzyme Conjugates,” in Immunobiology of Peptides and Proteins, vol. VI, pp. 97-105 (1991); as well as U.S. Pat. No. 6,436,437 and references cited therein.

Transportable peptides also can be conjugated to hydrophilic drugs, e.g., hydrophilic neuropeptides, which, alone, may be transportable across the blood-brain barrier only at a very low rate. The resulting conjugates are transported into the brain at a much higher rate than the drug alone, providing an effective means for introducing hydrophilic species into the brain through the blood-brain barrier. See, for example, U.S. Pat. No. 4,902,505. In order to increase transport across the blood-brain barrier, the prodrugs can also include R groups selected from the group consisting of docosohexaenoic acid, a transferrin receptor binding antibody, cationized albumin, Met-enkephalin, lipoidal forms of dihydropyridine, and cationized antibodies. See, for example, U.S. Pat. No. 6,627,601.

Other moieties can be conjugated to the prodrug to generally enhance drug delivery across epithelial tissues, including the skin, gastrointestinal tract, pulmonary epithelium, and the like, as well as the blood brain barrier. For example, R can be a moiety including guanidino or amidino side-chain groups, and/or delivery-enhancing polymers such as poly-arginine molecules. See, for example, U.S. Pat. No. 6,593,292.

The prodrugs can also include R moieties that allow the drugs to be specifically targeted to cell types or particular tissues, or to particular systems within an organism. Peptide-based compounds useful for this purpose are described, for example, in U.S. Patent Application Publication No. 2003/0149235. Such peptide-based compounds also can be used as targeting vectors that bind to receptors associated with angiogenesis. See U.S. Patent Application Publication Nos. 2003/0176639; and 2003/0194373. As particular examples, the botulinum neurotoxin heavy chain can be used as a moiety for targeting drugs to motor neurons (see, for example, U.S. Patent Application Publication No. 2003/0147921); and avidin-type molecules can be used to target the prodrug to liver cells and cells of the reticuloendothelial system (see, for example, U.S. Pat. No. 6,638,508).

The prodrugs can also be targeted to tumor-associated or inflammatory disease by conjugating the prodrug to certain polypeptides. See U.S. Patent Application Publication No. 2003/0166914 (methods of targeting cells expressing CD33-like polypeptides). Other vascular targeting moieties are disclosed in U.S. Patent Application Publication No. 2003/0149003 (stilbenoid compounds). Lectins (wheat germ agglutinin) can also be used for specific targeting, e.g., for delivery to colon carcinoma cells. See Wirth M, et al., Pharm Res.15(7):1031-7 (1998). Ester-type drug conjugates of beta-cyclodextrin can serve as a colon-targeting prodrugs. See Hirayama F., et al., Pharm Pbarmacol. 48(1):27-31(1996). Tumor vasculature can also be targeting using VEGF conjugates. See Ramakrishnan S., et al., Methods Mol. Biol. 166:219-34(2001).

The foregoing examples of conjugated moieties (R) for specific purposes is intended as illustrative of the invention and should not be taken as limiting in any way. One skilled in the art will recognize that suitable moieties for conjugation to achieve particular functionality will be possible within the bounds of the chemical conjugation mechanisms disclosed and claimed herein. Accordingly, additional moieties can be selected and used according to the principles of the invention as disclosed herein.

5.3 Synthesis Methods

The prodrugs described herein can be prepared by coupling an NH moiety present in the biologically active agent, or in a biologically active agent modified to include such an NH moiety, with an activated moiety including a —C(O)OR fragment or with an activated moiety that permits later attachment of an R or OR fragment, as appropriate.

Some biologically active agents already include an amide, thioamide, imide, thioimide, urea, thiourea, carbamate, thiocarbamate, sulfonamide or sulfonamide moiety that includes a reactive NH group capable of being coupled to the remainder of the prodrug (i.e., the —C(X)XR, preferably —C(O)OR, portion of the drug), or a synthetic precursor thereof. Other biologically active agents need to be modified to include a reactive NH group. For example, some biologically active agents, as discussed above, include hydroxyl, thiol, carboxylic acid or sulfonic acid groups which must be modified so that the biologically active agent includes an NH group. In still other embodiments, a biologically active agent already includes a reactive NH group, but it is desirable to maintain that NH group, for example, so that the activity of the biologically active agent is retained in the prodrug. In such embodiments, it may be desirable to protect the NH group using a protecting group, and possibly to modify the molecule to include an NH group at another position.

Where the biologically active agent includes a thiol group that is to be modified to form the prodrug, the thiol group can first be converted to a functional group that includes an NH group, such as a thiocarbamate (QSC(O)NHR′) (where Q is the remainder of the biologically active agent, D).

Where the biologically active agent includes a hydroxyl group that is to be modified to form the prodrug, the hydroxyl group can first be converted to a functional group that includes an NH group, such as a carbamate (-QOC(O)NHR′).

Where the biologically active agent includes a carboxylic acid group that is to be modified to form the prodrug, the carboxylic acid group can first be converted to a functional group that includes an NH group, such as an imide (QC(O)NHC(O)R′), amide (QC(O)NHR′), and the like.

Where the biologically active agent includes an amine group that is to be modified to form the prodrug, the amine group can first be converted to a functional group that includes an NH group, such as a carbamate (—OC(O)NHQ-), thiocarbamate (—OC(S)NHQ-), urea (-QNHC(O)NHR′, -QNHC(O)NHQ, -QNHC(O)NHR), thiourea (-QNHC(S)NHR′, -QNHC(S)NHQ, -QNHC(S)NHR), and the like.

It may be desirable to protect the NH groups with various protecting groups at certain stages of synthesis, and in these embodiments, R′can be further defined as a protecting group, such as t-boc or other suitable protecting group for a NH moiety, as such are known in the art.

Once the biologically active agent is identified which has an active NH group, or which is modified to include an active NH group, the NH group can be coupled with the remainder of the prodrug, or a synthetic precursor thereof. By synthetic precursor thereof, it is meant that D can be coupled in a first step to form compounds of formula D-C(═X)-activating group (such as a halogen), which can then be reacted with a compound of formula R-OH (or R—SH or R—NHR′) to form the compounds of Formula I.

Either the biologically active agent, D, with its active NH group, or a compound comprising R, can be activated such that D reacts with the compound comprising R to form a hydrolyzable carbamate functional group (or similar functional group in those compounds of Formula I where X is S or NR′) between the NH group on D (i.e., from the amide, thioamide, imide, thiomide, urea, thiourea, carbamate, thiocarbamate, sulfonamide, or sulfonamide group) and the compound comprising R. Following this coupling step, a prodrug is formed having a hydrolyzable carbamate-containing moiety.

The activated biologically active agent can be prepared using any of the various methods understood by those skilled in the art. In some embodiments, the biologically active agent is contacted with an activating agent such as a haloformyl halide under conditions sufficient to provide the activated biologically active agent. Those skilled in the art will understand the sufficient conditions, including suitable solvents, suitable reaction times, and suitable temperatures for performing this reaction.

In one embodiment, a biologically active agent that includes, or is modified to include, a functional group selected from the group consisting of amide, thioamide, imide, thoimide, urea, thiourea, carbamate, thiocarbamate, sulfonamide, or sulfonimideis dissolved in an appropriate (and preferably aprotic and anhydrous) solvent, and a suitable tertiary amine base, such as triethylamine, is added. The reaction mixture is cooled (for example, to 0° C.), and phosgene, diphosgene, or triphosgene is added. After the addition is complete, the reaction mixture can typically be allowed to warm to room temperature and stirred for an additional length of time (which, depending on the scale of the reaction, can be several hours to several days). After the resulting haloformate has formed, it can be reacted with a compound of formula R-OH (the reaction typically takes a few hours to complete) to form a compound of Formula I. The insoluble trialkylamine hydrochloride can be filtered out, or otherwise removed by washing the reaction mixture with an appropriate aqueous solvent, such as water or buffered water, to dissolve the trialkylamine hydrochloride. The product can be isolated, for example, by removing the solvent and distilling the product, by crystallization and filtration or centrifugation, or other means known to those of skill in the art. In some embodiments, the compounds can be purified using column chromatography, for example, using SiO₂: ethyl acetate/methanol, 90:10), to provide the desired product.

The compound that includes the R moiety (and a hydroxyl, thiol or amine group that is coupled to the remainder of the prodrug) can be provided by various methods as will be understood by those skilled in the art. In some embodiments, the compound comprising an R moiety has a reactive moiety such as hydroxyl or carboxyl, which is contacted with an activating agent such as a haloformyl halide, N-hydroxy succinimide, p-nitrophenyl, 3,4-dichlorophenyl, or 1-benzotriazolyloxy under conditions sufficient to provide the activated compound. The compound comprising an R moiety and having a reactive moiety can be various compounds that are desirable for conjugating with a biologically active agent to provide a prodrug of the present invention such as, but not limited to, those described in U.S. Pat. No. 4,179,337 to Davis et al.; U.S. Pat. No. 5,567,422 to Greenwald; U.S. Pat. No. 5,359,030 to Ekwuribe; U.S. Pat. No. 5,438,040 to Ekwuribe, U.S. Pat. No. 5,681,811 to Ekwuribe, U.S. Pat. No. 6,309,633 to Ekwuribe et al., and U.S. Pat. No. 6,380,405 to Ekwuribe et al.

For example, the oligomer can be a non-polydispersed oligomer as described in U.S. patent application Ser. No. 09/873,731 filed Jun. 4, 2001 by Ekwuribe et al. entitled “Methods of Synthesizing Substantially Monodispersed Mixtures of Polymers Having Polyethylene Glycol Mixtures”; U.S. patent application Ser. No. 09/873,797 filed Jun. 4, 2001 by Ekwuribe et al. entitled “Mixtures of Drug-Oligomer Conjugates Comprising Polyalkylene Glycol, Uses Thereof, and Methods of Making Same”; and U.S. patent application Ser. No. 09/873,899 filed Jun. 4, 2001 by Ekwuribe et al. entitled “Mixtures of Insulin Drug-Oligomer Conjugates Comprising Polyalkylene Glycol, Uses Thereof, and Methods of Making Same.” Those skilled in the art will readily understand the conditions sufficient for providing the activated compound(s). In general, those compounds having a reactive moiety are contacted with a suitable activating agent in a suitable solvent (aqueous or organic), for a suitable amount of time, at a suitable temperature as will be understood by those skilled in the art. For example, M-PEGₙ—OH (n is from 2 to 20) can be added dropwise to a cooled 20% phosgene solution in a solvent such as toluene. When the reaction is complete (for example, about 30 minutes at 0° C. and an additional 3 hours at room temperature), the excess phosgene and toluene can be distilled off under vacuum and the resultant activated compound, a chloroformate, can be used directly in the next step (with the compound structure optionally verified, for example, by IR spectroscopy).

Those skilled in the art will understand the conditions sufficient to couple the biologically active agent with the activated compound comprising an R moiety to provide a prodrug having a hydrolyzable carbamate moiety. In general, the biologically active agent is contacted with the activated compound comprising an R moiety in the presence of a suitable solvent. A suitable solvent will be one that can solubilize the biologically active agent and the compound comprising an R moiety to an extent that allows the compounds to react with one another. Suitable solvents include, but are not limited to, water-soluble and organic solvents. Examples of such organic solvents include C₁-C₄ alcohols such as methanol, ethanol, 1-propanol, 2-propanol, and butanol, as well as solvents such as acetone, tetrahydrofuran, acetonitrile, dimethyl formamide and dimethylsulfoxide.

The biologically active agent and the activated compound comprising an R moiety are preferably contacted for an amount of time and under conditions sufficient to provide a desirable yield of the prodrug.

Those skilled in the art will understand the conditions sufficient to couple the activated biologically active agent with the compound comprising an R moiety to provide a prodrug having a hydrolyzable carbamate moiety. In general, the activated biologically active agent is contacted with the compound comprising an R moiety in the presence of a suitable solvent. A suitable solvent will be one that can solubilize the activated biologically active agent and the compound comprising an R moiety to an extent that allows the compounds to react with one another. Suitable solvents include, but are not limited to, water-soluble and organic solvents. Examples of such organic solvents include C₁-C₄ alcohols such as methanol, ethanol, 1-propanol, 2-propanol, and butanol, as well as solvents such as acetone, tetrahydrofuran, acetonitrile, dimethyl formamide and dimethylsulfoxide.

The activated biologically active agent and the compound comprising an R moiety are preferably contacted for an amount of time that will provide a desirable yield of the prodrug.

Variations on the disclosed general synthetic methods will be readily apparent to those of ordinary skill in the art and are deemed to be within the scope of the present invention.

5.3.1.1 Formation of Pharmaceutically Acceptable Salts

In some embodiments, the prodrug is provided as a pharmaceutically acceptable salt.

Pharmaceutically acceptable salts are salts that retain the desired biological activity of the parent compound and do not impart undesired toxicological effects. Examples of such salts are (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (b) salts formed from elemental anions such as chlorine, bromine, and iodine. In preferred embodiments, the pharmaceutically acceptable salt is formed with hydrochloric acid or acetic acid.

5.4 Pharmaceutical Compositions

Pharmaceutical compositions including the prodrugs described herein can be prepared. Such 15. compositions typically include the prodrug in combination with, or in admixture with, a pharmaceutically acceptable carrier. The carrier must, of course, be acceptable in the sense of being compatible with any other ingredients in the pharmaceutical composition and should not be deleterious to the patient. The carrier may be a solid or a liquid, or both, and is preferably formulated with the prodrug as a unit-dose formulation, for example, a tablet, which may contain from about 0.01 20 or 0.5% to about 95% or 99% by weight of the prodrug. The pharmaceutical compositions may be prepared by any of the well-known techniques of pharmacy including, but not limited to, admixing the components, optionally including one or more accessory ingredients.

The pharmaceutical compositions according to embodiments of the present invention include those suitable for oral, rectal, nasal, topical, inhalation (e.g., via an aerosol) buccal (e.g., sub-lingual), vaginal, parenteral (e.g., subcutaneous, intramuscular, intradermal, intraarticular, intrapleural, intraperitoneal, intracerebral, intraarterial, or intravenous), topical. (i.e., both skin and mucosal surfaces, including airway surfaces) and transdermal administration, although the most suitable route in any given case will depend on the nature and severity of the condition being treated and on the nature of the particular prodrug which is being used.

Pharmaceutical compositions suitable for oral administration may be presented in discrete units, such as capsules, cachets, lozenges, or tables, each containing a predetermined amount of the prodrug; as a powder or granules; as a solution or a suspension in an aqueous or non-aqueous liquid; or as an oil-in-water or water-in-oil emulsion. Such formulations may be prepared by any suitable method of pharmacy that includes the step of bringing into association the prodrug and a suitable carrier (which may contain one or more accessory ingredients as noted above). In general, the pharmaceutical composition according to embodiments of the present invention are prepared by uniformly and intimately admixing the prodrug with a liquid or finely divided solid carrier, or both, and then, if necessary, shaping the resulting mixture. For example, a tablet may be prepared by compressing or molding a powder or granules containing the prodrug, optionally with one or more accessory ingredients. Compressed tablets may be prepared by compressing, in a suitable machine, the mixture in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Pharmaceutical compositions suitable for buccal (sub-lingual) administration include lozenges comprising the prodrug in a flavored base, usually sucrose and acacia or tragacanth; and pastilles comprising the prodrug in an inert base such as gelatin and glycerin or sucrose and acacia.

Pharmaceutical compositions according to embodiments of the present invention suitable for parenteral administration comprise sterile aqueous and non-aqueous injection solutions of the prodrug, which preparations are preferably isotonic with the blood of the intended recipient. These preparations may contain anti-oxidants, buffers, bacteriostats and solutes which render the composition isotonic with the blood of the intended recipient. Aqueous and non-aqueous sterile suspensions may include suspending agents and thickening agents. The compositions may be presented in unit\dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. For example, an injectable, stable, sterile composition comprising a prodrug in a unit dosage form in a sealed container may be provided. The prodrug is provided in the form of a lyophilizate which is capable of being reconstituted with a suitable pharmaceutically acceptable carrier to form a liquid composition suitable for injection thereof into a subject. The unit dosage form typically comprises from about 10 mg to about 10 grams of the prodrug. When the prodrug is substantially water-insoluble, a sufficient amount of emulsifying agent which is physiologically acceptable may be employed in sufficient quantity to emulsify the prodrug in an aqueous carrier. One such useful emulsifying agent is phosphatidyl choline.

Pharmaceutical compositions suitable for rectal administration are preferably presented as unit dose suppositories. These may be prepared by admixing the prodrug with one or more conventional solid carriers, for example, cocoa butter, and then shaping the resulting mixture.

Pharmaceutical compositions suitable for topical application to the skin preferably take the form of an ointment, cream, lotion, paste, gel, spray, aerosol, or oil. Carriers which may be used include petroleum jelly, lanolin, polyethylene glycols, alcohols, transdermal enhancers, and combinations of two or more thereof.

Pharmaceutical compositions suitable for transdermal administration may be presented as discrete patches adapted to remain in intimate contact with the epidermis of the recipient for a prolonged period of time. Compositions suitable for transdermal administration may also be delivered by iontophoresis (see, for example, Pharmaceutical Research 3 (6):318 (1986)) and typically take the form of an optionally buffered aqueous solution of the prodrug. Suitable formulations comprise citrate or bis\tris buffer (pH 6) or ethanol/water and contain from 0.1 to 0.2M active ingredient.

5.5 Methods of Treatment Using the Prodrugs

Subjects taking or inclined to take the parent drug can alternatively (or additionally) take the prodrugs described herein. For example, patients suffering from disorders that are conventionally treated using the parent drugs can be treated using an effective amount of the prodrugs described herein. Advantageously, where such drugs were previously only administrable via injection or intraveneous administration, the prodrugs can be administered via inhalation or, more preferably, oral administration.

In one embodiment, the invention provides a method of delivering a biologically active agent to a subject, wherein the biologically active agent is orally administered as a component of a prodrug of the invention, a portion of the orally administered prodrug survives intact in the GI tract and traverses the intestinal wall to enter the bloodstream, and after leaving the GI tract, some or all of the prodrug is hydrolyzed in vivo to yield a pharmaceutically acceptable amount of the biologically active agent. The hydrolysis may, for example, take place in the bloodstream or in the liver. In this method, the prodrug enhances the oral bioavailability of the orally administered biologically active agent relative to the oral bioavailability of a corresponding orally administered unconjugated biologically acitive agent.

The effective amount of any prodrug, the use of which is in the scope of present invention, will vary somewhat from prodrug to prodrug, and patient to patient, and will depend upon factors such as the age and condition of the patient and the route of delivery. Such dosages can be determined in accordance with routine pharmacological procedures known to those skilled in the art. As a general proposition, a dosage from about 0.1 to about 50 mg/kg will have therapeutic efficacy, with all weights being calculated based upon the weight of the prodrug. Toxicity concerns at the higher level may restrict intravenous dosages to a lower level such as up to about 10 mg/kg, with all weights being calculated based upon the weight of the active base. A dosage from about 10 mg/kg to about 50 mg/kg may be employed for oral administration. Typically, a dosage from about 0.5 mg/kg to 5 mg/kg may be employed for intramuscular injection. The frequency of administration is usually one, two, or three times per day or as necessary to control the condition. The duration of treatment depends on the type of condition being treated and may be for as long as the life of the patient.

Suitable subjects to be treated according to the present invention include, but are not limited to, avian and mammalian subjects, preferably mammalian. Mammals according to the present invention include but are not limited to canine, felines, bovines, caprines, equines, ovines, porcines, rodents (e.g. rats and mice), lagomorphs, primates, humans, and the like, and encompass mammals in utero. Any mammalian subject in need of being treated according to the present invention is suitable. Human subjects are preferred. Human subjects of both genders and at any stage of development (ie., neonate, infant, juvenile, adolescent, adult) can be treated according to the present invention.

Illustrative avians according to the present invention include chickens, ducks, turkeys, geese, quail, pheasant, ratites (e.g., ostrich) and domesticated birds (e.g., parrots and canaries), and include birds in ovo.

### 6 EXAMPLES

All starting materials used in the procedures described herein are either commercially available, can be prepared by methods known in the art using commercially available starting materials, or have been obtained from Bristol-Myers Squibb Company (Princeton, N.J.).

6.1 Synthesis of C10-PEG5 Oligomer

Decanoic acid (1.21 g, 7.00 mmol) was dissolved in neat oxalyl chloride (5 mL) and the reaction mixture was stirred at room temperature for 1 hour. The excess oxalyl chloride was removed in vacuo to give the desired acid chloride as a clear, colorless oil in 100% yield (1.34 g). Analysis by IR showed complete conversion of the carboxylic acid to the acid chloride: IR (NaCl) 2921, 2856, 1801, 1465, 1401, 1130, 1000, 953, 923 cm⁻¹.

Azeotropically dried pentaethylene glycol (5.00 g, 21.0 mmol) was dissolved in anhydrous THF (20 mL) and triethylamine (1.12 mL, 8.05 mmol) was added with stirring. Decanoic acid chloride (1.34 g, 7.00 mmol) in anhydrous THF (10 mL) was added dropwise to the PEG solution. Upon addition, a white precipitate of triethylamine hydrochloride was formed. The reaction mixture was stirred at room temperature for 17 hours and the THF was removed under reduced pressure resulting in a yellow oil. Dichloromethane (25 mL) was added to the oil and the resulting organic solution was washed with water (2×25 mL) and brine (2×25 mL). The organic layer was then dried over MgSO₄, filtered, and the solvent removed under reduced pressure to give a pale yellow oil. Purification by column chromatography (SiO₂: ethyl acetate, 100%) gave the desired oligomer as a clear, pale yellow oil (1.17 g, 42.5%). IR (NaCl): 3470 (br), 2904, 1731, 1460, 1354, 1254, 1112, 947 cm⁻¹; MS (FAB in NBA) m/z 393 (M+H)⁺. See scheme below.

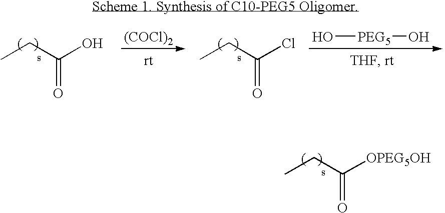

6.2 Synthesis of C6-PEG5 Oligomers

Hexanoic acid (1.63 g, 14.0 mmol) was dissolved in neat oxalyl chloride (SmL) and the reaction mixture was stirred at room temperature for 1 hour. The excess oxalyl chloride was removed in vacuo to give the desired acid chloride as a clear, colorless oil in 100% yield (2.54 g). Analysis by IR showed complete conversion of the carboxylic acid to the acid chloride: IR (NaCl) 2919, 2872, 1805, 1467, 1401, 1122, 970, 917 cm⁻¹.

Azeotropically dried pentaethylene glycol (10.0 g, 42.0 mmol) was dissolved in anhydrous THF (20 mL) and triethylamine (2.15 mL, 15.4 mmol) was added with stirring. Hexanoic acid chloride (2.54 g, 14.0 mmol) in anhydrous THF (10 mL) was added dropwise to the PEG solution. Upon addition, a white precipitate of triethylamine hydrochloride was formed. The reaction mixture was stirred at room temperature-for 17 hours and the THF was removed under reduced pressure resulting in a yellow oil. Dichloromethane (25 mL) was added to the oil and the resulting organic solution was washed with water (2×25 mL) and brine (2×25 mL). The organic layer was then dried over MgSO₄, filtered, and the solvent removed under reduced pressure to give a pale yellow oil. Purification by column chromatography (SiO₂: ethyl acetate, 100%) gave the desired oligomer as a clear, pale yellow oil (2.09 g, 51.0%). IR (NaCl): 3452 (br), 2886, 1737, 1460, 1354, 1248, 1130, 953 cm⁻¹; MS (FAB in NBA) m/z 337 (M+H)⁺.

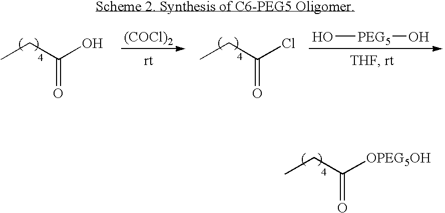

6.3 Synthesis and Purification of Dilantin® Prodrug

Synthesis of a Dilantin® prodrug was accomplished according to the procedure described herein. The activated oligomer was prepared by adding 5 g (30 mmol) of 2-(2-Ethoxy-ethoxy)-ethanol dropwise into a 30 mL cooled solution of 20% phosgene in toluene. See scheme below. The reaction mixture was stirred near 0° C. for about 30 minutes. The ice bath was removed and the reaction mixture was stirred continuously at room temperature for another 3 hours. Excess phosgene and toluene were then distilled off under vacuum (T=50° C.), and the resultant chloroformate, which was a viscous oil, was used for the next step without purification.

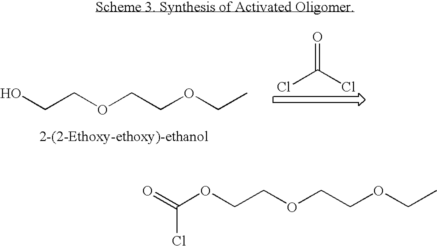

0.2 mL of the chloroformate solution in anhydrous acetonitrile was added dropwise under nitrogen to a precooled stirred mixture of diphenylhydantoin (100 mg, 0.397 mmol) in anhydrous acetonitrile and TEA (50 mg, 0.495 mmol). See scheme below. After all oligomer had been introduced, the reaction mixture was stirred at room temperature for 1-2 hours. Reaction was followed by HPLC (reverse phase C18 column) and TLC (developing solvent ethyl acetate or chloroform/methanol 90/10%). Acetonitrile was then removed and the reaction mixture was reconstituted with chloroform and organic solution, washed with water, dried over MgSO₄, filtered, concentrated in rotovap, and dried under vacuum. Crude compound was purified by chromotagraphy using chloroform/methanol (90/10%) and ethyl acetate. FABMS ⁺H 443 HPLC, H¹ NMR of phenyloin prodrug and parent phenyloin, and COSY of the Dilantin prodrug were performed.

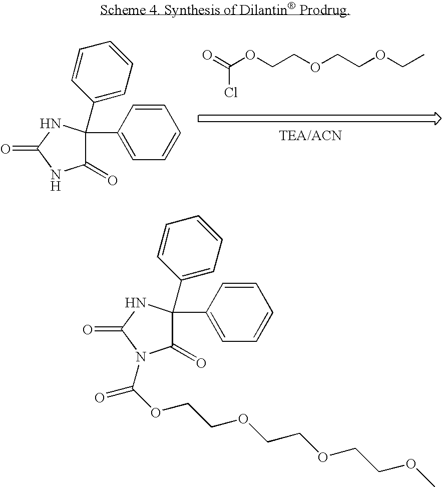

6.4 Preparation of hex-PEG₅-Dilantin® Prodrug

The procedure of Example 6.3 is repeated substituting the oligomer of Example I for the PEG3 to provide a hex-PEG5-Dilantin® prodrug.

6.5 Preparation of deca-PEG₅-Dilantin® Prodrug

The procedure of Example 6.3 is repeated substituting the oligomer of Example 2 for the PEG3 to provide a Deca-PEG₅-Dilantin® prodrug.

6.6 Stability of Dilanfin® (phenyloin) Prodrug at pH 2.0 and in Rat Plasma

Aliquots of a stock solution of Dilantin®-PEG3 conjugate in acetonitrile were spiked into 0.06M phosphate buffer, pH 2.0 (2 μl stock solution in 998 μl buffer). The sample was incubated in a water bath at 37° C. At time points one hour apart, a portion of the sample was removed and submitted for immediate analysis by RP-HPLC. Results are shown in Table 1, below.

Also using a stock solution of Dilantin®-PEG3 conjugate in acetonitrile, the following spike controls were prepared in rat plasma: 1 μg/ml, 750 ng/ml, 500 ng/ml and 250 ng/ml. After vortexing, 500 mL of acetonitrile was added. Each sample was centrifuged immediately and the resultant supernatant was removed and the pellet discarded. The supernatant was concentrated and reconstituted with 100 mL of 30% acetonitrile. Prior to transferring samples to HPLC vials, the samples were centrifuged for 15 minutes at 14,000 rpm at 2° C. to remove particulates. Samples were submitted for RP-HPLC anaylsis. Such analysis revealed that in all cases, the extracted samples showed only the presence of phenyloin, the parent compound. Thus, near complete plasma hydrolysis of the conjugate may occur within 1 minute. Studies were repeated a second day to confirm results.

TABLE 1
Chemical hydrolysis of Dilantin ® -conjugate
(PEG-3) at a pH of about 2.

| Time, | Dilantin ® -PEG3 |
|---|---|
| Hours | (Remaining), % |
| 0 | 100 |
| 2 | 96.79 |
| 3 | 94.99 |

6.7 Solubility of Dilantin®-PEG3 Conjugate

5,5-diphenylhydrantoin and Dilantin®-PEG3 conjugate prodrug solutions were made in acetonitrile with concentrations of 1.12 mg/mL and 0.8 mg/mL, respectively. These compounds had 100% solubility in acetonitrile and served as standards.

Solutions of 5,5-diphenylhydrantoin and Dilantin®-PEG3 conjugate were then made in deionized water at concentrations of 0.99 mg/mL and 0.93 mg/mL, respectively. These solutions were mixed well by vortexing. 1 mL aliquots were taken and spun by centrifuge at 5000 rpm for five minutes. The resultant supemant was taken and analyzed by HPLC for solution concentration. The concentration of 5,5-diphenylhydrantoin was 4 μg/mL. The concentration of Dilantin®-PEG3 conjugate was 160 μg/mL. Thus, the Dilantin®-PEG3 conjugate was more soluble in water than the native drug.

6.8 Synthesis of a Prodrug of 1,4-Dihydro-[3-[[[[3-[4-(3-methoxyphenyl)-1-piperidinyl]propyl]amino]carbonyl]amino]phenyl]-2,6-dimethyl-3,5-pyridinedicarboxylic acid, dimethyl ester

1,4-Dihydro-[3-[[[[3-[4-(3-methoxyphenyl)-1-piperidinyl]propyl]amino]carbonyl]amino]phenyl]-2,6-dimethyl-3,5-pyridinedicarboxylic acid, dimethyl ester (1.00 g, 1.69 mmol) (note: this compound is also sometimes referred to below as the parent compound, native compound or NC) was dissolved in anhydrous CH₂Cl₂ (40 mL) and triethylamine (0.260 mL, 1.86 mmol) through stirring. The reaction mixture was cooled to 0° C. in an ice bath. p-Nitrophenylchloroformate (1.13 g, 5.58 mmol) in anhydrous CH₂Cl₂ (10 mL) was added dropwise to the stirring drug solution. Upon complete addition, the reaction mixture was allowed to warm to room temperature and stirred for 18 hours. The reaction progress was monitored by RP-HPLC. Once complete reaction of the chloroformate had taken place, ethyl glycolate (0.481 mL, 5.58 mmol) in anhydrous CH₂Cl₂ (3 mL) was added to the stirring solution at room temperature. The reaction was complete after 2 hours, at which point the solvent was removed under reduced pressure to give a bright yellow oil. Purification by column chromatography (SiO₂: ethyl acetate/methanol, 90:10) gave the desired product in 73% yield (0.890 g): HPLC: retention time=21.589 min, purity=>99%; MS (FAB in NBA) m/z 721 (M+H)⁺.

6.9 Synthesis of a Prodrug of 1,4-Dihydro-[3-[[[[3-[4-(3-methoxyphenyl)-1-piperidinyl]propyl]amino]carbonyl]amino]phenyl]-2,6-dimethyl-3,5-pyridinedicarboxylic acid, dimethyl ester

Five grams (30 mmol) of 2-(2-Ethoxy-ethoxy)-ethanol was added dropwise into 30 ML of cooled solution of 20% phosgene in toluene. The reaction mixture was stirred near 0° C. for about 30 minutes. The ice bath was removed and the reaction mixture was stirred at room temperature for another 3 hours. Excess phosgene and toluene were then distilled off under vacuum (T=50° C.), and the resultant chloroformate, which was a viscous. oil, was used for the next step without purification. Analysis by IR showed complete conversion of the alcohol to the chloroformate.

The chloroformate (1 g, 4.42 mmol) was dissolved in anhydrous methylene chloride and TEA (520 mg, 5.15 mmol) added. Then the mixture was cooled with an ice bath and a solution of 1,4-Dihydro-[3-[[[[3-[4-(3-methoxyphenyl)-1-piperidinyl]-propyl]amino]carbonyl]amino]phenyl]-2,6-dimethyl-3,5-pyridinedicarboxylic acid, dimethyl ester (1 g, 1.60 mmol) in methylene chloride was added dropwise into the chloroformate solution under nitrogen. The reaction mixture was stirred for about 3 hours. The reaction mixture was then washed with deionized water, dried over MgSO₄, filtered and concentrated via rotovap to afford a crude compound. The crude prodrug compound was chromatographed on silica gel column using chloroform/methanol (85%/15%) as eluting solvent. Fractions containing the product were combined, concentrated, and dried via vacuum (95% yield.) The purified compound was analyzed by FABS: m/e 782/M⁺H, HPLC, NMR.

The prodrugs were converted into hydrochloride salt according to the following procedure: Prodrug (free base) was dissolved in a minimal amount of anhydrous THF. Then HCl in diethyl ether was added. The mixture was stirred for 1 hour. Salt was collected and dried via vacuum overnight.

6.10 Stability at pH 2 and Oral Absorption of Prodrugs of 1,4-Dihydro-[3-[[[[3-[4-(3-methoxyphenyl)-1-piperidinyl]propyl]amino]carbonyl]amino]phenyl]-2,6-dimethyl-3,5-pyridinedicarboxylic acid, dimethyl ester

Five rats were dosed for each 1,4-dihydro-[3-[[[[3-[4-(3-methoxyphenyl)-1-piperidinyl]propyl]amino] carbonyl]amino]phenyl]-2,6-dimethyl-3,5-pyridinedicarboxylic acid, dimethyl ester pro-drug and the parent compound which were formulated in Cremophor-EL/Ethanol/Water, 10:10:80 for all compounds. 100 μL of plasma was extracted with 500 μL of acetonitrile, vortexed and centrifuged. The supernatant was removed, evaporated to dryness, and then reconstituted with 100 μL of 30% methanol. The reconstituted samples were then centrifuged to remove particulates. The supernatant was removed and transferred to HPLC vials for detection and quantitation of the parent compound by LC/MS/MS. All AUC values for the parent compound have been calculated through 24 hours. Rats were dosed at 25.6 mg/kg (p.o.). See Table 2 below. Percent yield, MS, and HPLC purity as reported in Table 2 refer to the isolated yield from the synthesis of the prodrug compounds and the characterization of that compound.

TABLE 2
Chemical stability and Oral Absorption of Carbamate Prodrugs

| | | | | | | HPLC | Percent | |
|---|---|---|---|---|---|---|---|---|
| Compound | Compound | | pH 2 | Oral | MS | Purity | Yield | |
| I.D. # | Structure | Salt | Stability | Absorption | (M + H) | (%) | (%) | |
| Native drug | R = H | Lactate | >2 h | 0.1% | m/z 591 | | | |
| 1 | 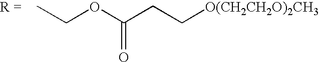 | — | None | Not Determined | Not Definitive | — | — | |
| 2 | 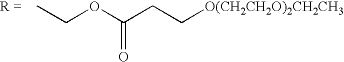 | — | None | Not Determined | Not Definitive | — | — | |
| 3 | 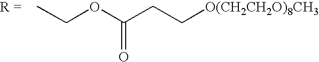 | — | None | Not Determined | Not Definitive | — | — | |
| 4 | 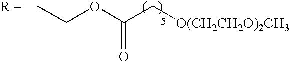 | — | None | Not Determined | Not Definitive | — | — | |
| 5 | 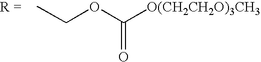 | — | None | Not Determined | Not Definitive | — | — | |
| 6 | 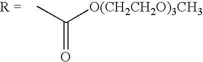 | — | >2 h | Not Detected | m/z 782 | >99% | 95% | |
| 7 | 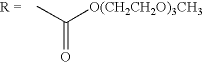 | — | >2 h | Not Detected | m/z 870 | >99% | 92% | |
| 8 | 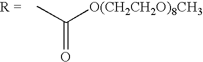 | — | >2 h | Not Detected | m/z 1001 | >99% | 54% | |
| 9 | 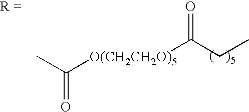 | HCl | >2 h | Not Detected | | m/z 954 | >99% | 80% |
| 10 | 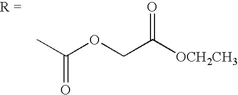 | HCl | Not Determined | Not Detected | m/z 721 | >99% | 73% | |
| | | | | Oral | | | | |
| | | | | Absorption | | HPLC | Percent | |
| Compound | Compound | | pH 2 | (Relative | MS | Purity | Yield | |
| I.D. # | Structure | Salt | Stability | AUCs) | (M + H) | (%) | (%) | |
| 11 | 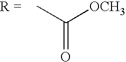 | HCl | >2 h | 275 (2 h) dose: 150 mg/kg | m/z 649 | >99% | 94% | |
| 12 | 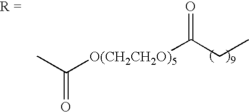 | HCl | Not Determined | Not Detected | m/z 1009 | >99% | 95% | |
| 13 | 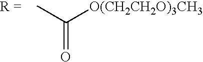 | HCl | >2 h | 95 (24 h) dose: 25.6 mg/kg | m/z 782 | >99% | 95% | |
| 14 | 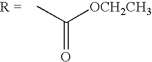 | HCl | >2 h | 1900 (24 h) dose: 150 mg/kg | m/z 663 | >99% | 90% | |
| 15 | 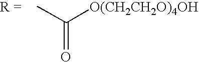 | HCl | Not Determined | Minimal | m/z 783 | >99% | 65% | |

| 16 | 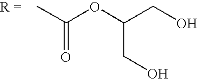 | HCl | Not Determined | Minimal | m/z 709 | Not Determined | |
|---|---|---|---|---|---|---|---|
| 17 | 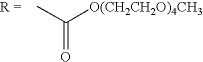 | HCl | >2 h | 90 (24 h) dose: 25.6 mg/kg | m/z 826 | >99% | 92% |
| 18 | 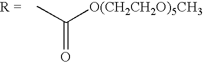 | HCl | >2 h | 763 (24 h) dose: 150 mg/kg | m/z 913 | >99% | 72% |
| 19 | 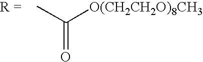 | HCl | >2 h | 1030 (24 h) dose: 150 mg/kg | m/z 1001 | >99% | 56% |
| 20 | 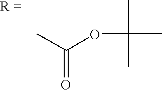 | HCl | Not Determined | Not Determined | m/z 691 | Not Determined | |
| 21 | 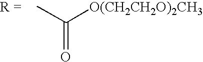 | HCl | >2 h | 130 (24 h) dose: 25.6 mg/kg | m/z 738 | >99% | 96% |
| 22 | 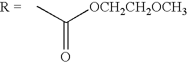 | HCl | >2 h | 102 (24 h) dose: 25.6 mg/kg | m/z 693 | >99% | 97% |

| | | | | | | |
|---|---|---|---|---|---|---|
| 23 | 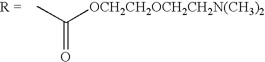 | HCl | Not Determined | Not Determined | m/z 751 | Not Determined |
| 24 | 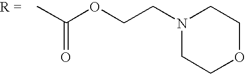 | HCl | Not Determined | Not Determined | m/z 749 | Not Deteremined |

| | | | | | |
|---|---|---|---|---|---|
| 25 | 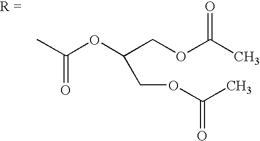 | HCl | Not Deteremined | Not Determined | Not Determined |

*R is coupled to anj amino group of the 1,4-Dihydro-[3-[[[[2-[4-(3-methoxyphenyl)-1-piperidinyl]-propyl]amino]carbonyl]amino]phenyl]-2,6-dimethyl-3,5-pyridinedicarboxylic acid, dimethyl ester.

In order to determine the stability of prodrugs of 1,4-dihydro-[3-[[[[3-[4-(3-methoxyphenyl)-1-piperdinyl]propyl]amino]carbonyl]amino]phenyl]-2,6-dimethyl-3,5-pyridinedicarboxylic acid, dimethyl ester at about a pH of 2 at 37° C. for about 2 hours, 2 μL aliquots of each 1,4-dihydro-[3-[[[[3-[4-(3-methoxyphenyl)-1-piperidinyl]propyl]-amino]carbonyl]amino]phenyl]-2,6-dimethyl-3,5-pyridinedicarboxylic acid, dimethyl ester prodrug in acetonitrile were spiked into 998 μL of 0.06M phosphate buffer, pH 2.0. The samples were incubated in a water bath at 37° C. for 2 hours. Each sample was then removed from the water bath and vortexed. 25 μL of control 1,4-dihydro-[3-[[[[3-[4-(3-methoxyphenyl 1-piperidinyl]propyl]amino]carbonyl]amino]phenyl]-2,6-dimethyl-3,5-pyridinedicarboxylic acid, dimethyl ester and each pro-drug (2 μL spiked into 998 μL of 30% acetonitrile kept at room temperature) were also analyzed by RP-HPLC. Each sample was subjected to exactly 2 hours of chemical hydrolysis and analyzed immediately by RP-HPLC. Table 3 below summarizes the pH 2.0 stability of 1,4-dihydro-[3-[[[[3-[4-(3-methoxyphenyl)-1-piperidinyl]propyl]amino]carbonyl]amino]-phenyl]-2,6-dimethyl-3,5-pyridinedicarboxylic acid, dimethyl ester prodrugs.

TABLE 3
pH 2.0 Stability of Prodrugs of 1,4-Dihydro-[3-[[[[3-
[4-(3-methoxyphenyl)-1-
piperidinyl]propyl]amino]carbonyl]amino]phenyl]-
2,6-dimethyl-3,5-pyridinedicarboxylic acid, dimethyl ester

| Pro-drug | % Area of Pro-drug | % Area NC |
|---|---|---|
| 13 | 100 | 0 |
| 17 | 100 | 0 |
| 21 | 100 | 0 |
| 22 | 100 | 0 |

6.11 Rat Plasma Stability of Selected Prodrugs

Using 1 mg/mL stock solutions of a prodrug described in Example 6.8 above in acetonitrile, 990 μL of rat plasma was spiked with 10 μL of conjugate. The plasma spikes were incubated at 37° C. 100 μL of each sample at each time-point was removed and the reaction was quenched with 500 μL of acetonitrile. Each sample was vortexed and centrifuged immediately and the resultant supernatant was removed, discarding the pellet. The supernatant was concentrated and reconstituted with 100 μL of 30% acetonitrile. Prior to transferring samples to HPLC vials, the samples were centrifuged for 15 minutes at 14000 rpm at 2° C. to remove particulates. 10 μL of 10 μg/mL standard and 75 μL of reconstituted sample were injected for RP-HPLC analysis.

The stability of the prodrugs was measured by analyzing samples at the following time-points: 0, 60, 120, 240, 360, and 480 minutes. Retention time, peak area, and UV spectrum were measured for each sample and standard. Conversion of the prodrug to the native drug and other metabolites is reported in Table 4 as a function of peak area. A Waters 2690 HPLC was used for sample analysis (column: Delta Pak C18 Guard Column, C18, 5 m, 2.1 mm×50 mm; Collected spectrum: 210-380 nm. Extracted chromatogram: 240 nm; Mobile Phase; 0.1% TFA in Water/0.1% TFA in acetonitrile; flow Rate: 0.5 mL/minute).

TABLE 4

| | | % comp. | % comp. | % comp. | % comp. | % comp. |
|---|---|---|---|---|---|---|
| Compound | % comp. 0′ | 60′ | 120′ | 240′ | 360′ | 480′ |
| 13 | 99.0 | 98.0 | 98.0 | 97.0 | 96.0 | 95.0 |
| 14 | 98.0 | 98.0 | 97.5 | 96.5 | 96.0 | 95.0 |
| 15 | 97.0 | 97.0 | 95.5 | 94.0 | 93.0 | 92.0 |
| 16 | 0 | 0 | 0 | 1 | 1 | 1 |
| 17 | 99.7 | 99.0 | 98.5 | 98.0 | 97.0 | 96.0 |
| 18 | 97.3 | 97.0 | 97.0 | 96.0 | 96.0 | 95.0 |
| 19 | 97.0 | 97.0 | 96.5 | 96.0 | 95.3 | 94.0 |

Compound 16 is unstable in rat plasma and conversion to the native urea drug compound is a function of plasma hydrolysis. Essentially 100% of the prodrug is converted to the native compound within 30 seconds of being introduced to rat plasma. The other prodrugs studied in this assay are stable in rat plasma. After 8 hours, only about 5% of the prodrug was converted to the native urea drug compound despite the previously seen hydrolysis in vivo after oral dosing. Hydrolysis of these compounds may occur in the liver.

The foregoing examples are illustrative of the present invention, and are not to be construed as limiting thereof. The invention is defined by the following claims, with equivalents of the claims to be included therein.

What is claimed is:

1. A prodrug having the following formula: 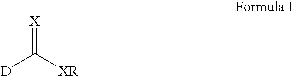
wherein:
X is O, S, or NR′;
R′is, individually, hydrogen, alkyl, substituted alkyl, aryl, substituted aryl, arylalkyl, substituted arylalkyl, alkylaryl, or substituted alkylaryl;
D is a biologically active agent comprising a functional group selected from the group consisting of urea, thiourea, amide, thioamide, imide, thioimide, carbamate, thiocarbamate, sulfonamide, and sulfonimide, phosphoramide, or which originally included a hydroxyl, amine, thiol, and/or carboxylic acid group, where such hydroxyl, thiol or carboxylic acid group has been modified to be in the form of a urea, thiourea, amide, thioamide, imide, thioimide, carbamate, thiocarbamate, sulfonamide, or sulfonamide group;
the linkage between D and the C═X moiety is through an —N— linkage, formed from an NH group present in the urea, thiourea, amide, thioamide, imide, thioimide, carbamate, thiocarbamate, sulfonamide, or sulfonamide group in D and the C═X moiety; and
R is a modifying moiety that provides the prodrug with one or more improved pharmaceutical characteristics selected from the group consisting of improved ability of the prodrug to pass through the GI tract and enter the blood stream; improved hydrophilicity, hydrophobicity, or amphiphilicity of the prodrug; improved solubility of the prodrug in aqueous environments or organic solvents; improved ability of the prodrug to cross cell membranes; improved ability of the prodrug to cross the blood-brain barrier; improved ability of the prodrug to target a certain receptor, cell, tissue, or organ; and improved pharmacokinetic profile of the prodrug.

2. The prodrug of claim 1, wherein the one or more improved characteristics comprises the ability of the prodrug to be orally delivered in a dosage that ultimately provides a pharmaceutically acceptable amount of the biologically active moiety, D, in systemic circulation.

3. The prodrug of claim 1, wherein the one or more improved characteristics comprises improved decreased degradation of of the biologically active agent component of the prodrug relative to unconjugated biologically active agent, D, at a pH of about 2 for less than about 2 hours.

4. The prodrug of claim 1, wherein R is a moiety that affects the solubility of D such that the prodrug is more soluble in plasma than the biologically active agent, D.

5. The prodrug of claim 1, wherein administration to a subject of an amount of the prodrug yields greater bioavailability of the biologically active agent than administration of unconjugated biologically active agent, D.

6. The prodrug of claim 1, wherein the biologically active agent component of the prodrug is more stable as a component of the prodrug than the biologically active agent from which the prodrug is derived in the presence of plasma, proteases, liver homogenate, acidic conditions, or basic conditions.

7. The prodrug of claim 1, having the formula: 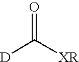
where D, X and R are as defined in claim 1.

8. The prodrug of claim 1, having the formula: 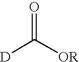
where D and R are as defined as in claim 1.

9. The prodrug of claim 1, wherein D is derived from a biologically active agent comprising a functional group selected from the group consisting of urea, thiourea, amide, thioamide, imide, thioimide, carbamate, thiocarbamate, sulfonamide, sulfonamide and phosphoramide.

10. The prodrug of claim 1, wherein D is derived from a biologically active agent comprising a thiol functional group that has been modified to be in the form of a thiocarbamate moiety.

11. The prodrug of claim 10, wherein the thiol-containing drug is selected from the group consisting of thiol-containing peptides and proteins, thiol-containing anti-inflammatory drugs, thiol-containing antirheumatic drugs, thiol-containing peptidomimetic inhibitors, thiol-containing angiotensin converting enzyme inhibitors, cysteine proteases, and thiol-containing antimicrobial heterocycles.

12. The prodrug of claim 10, wherein the thiol-containing drug is selected from the group consisting of glutathione, metallothiones, homo-cysteine, N-acetyl cysteine (NAC), D-penicillamine, captopril, 6-mercaptopurine, mercaprol, dimercaptopropanesulfonate, cathepsin B, cathepsin K, cathepsin L, capthepsin S, thioredoxin reductase, and thiol-containing transcription factors.

13. The prodrug of claim 1, wherein D is derived from a biologically active agent comprising a hydroxyl functional group that has been modified to be in the form of a carbamate moiety.

14. The prodrug of claim 1, wherein D is derived from a hydroxyl-containing drug selected from the group consisting of antineoplastic agents, anti-tumor agents, anti-viral and/or anti-tumor nucleosides, antibiotics and steroids.

15. The prodrug of claim 14, wherein the antitumor agents are selected from the group consisting of taxol, doxorubicin, bleomycin, vincristine (vinblastine), daunorubicin, and idarubicin.

16. The prodrug of claim 14, wherein the antiviral and/or anti-tumor nucleosides are selected from the group consisting of ddI(didanosine), ddC (zalcitabine), d4T (stavudine), FTC, lamivudine (3TC), 1592U89 (4-[2-amino-6-(cyclopropylamino)-9H-purin-9-yl]-2-cyclopentene-1-methanol), AZT (zidovudine), DAPD (D-2,6-diaminopurine dioxolane) and F-ddA.

17. The prodrug of claim 1, wherein D is derived from a biologically active agent comprising an amine functional group that has been modified to be in the form of a carbamate, thiocarbamate, urea, thiourea, amide, thioamide, sulfonamide or sulfonamide functional group.

18. The prodrug of claim 17, wherein the drug is selected from the group consisting of proteins, peptides, and compounds that bind to CNS receptors.

19. The prodrug of claim 1, wherein D is derived from a biologically active agent comprising a carboxylic acid functional group that has been modified to be in the form of an amide, thioamide, imide or thioimide moiety.

20. The prodrug of claim 1, wherein D is selected from the group consisting of phenyloin, droperidol, sulperidol, primidone, clonazepam, glipizide, glyburide, and tolbutamide.

21. The prodrug of claim 1, wherein R is a hydrophilic moiety.

22. The prodrug of claim 21, wherein the hydrophilic moiety is a polyalkylene glycol moiety, a sugar moiety, or a polysorbate moiety.

23. The prodrug of claim 21, wherein the polyalkylene glycol is selected from the group consisting of polyethylene glycol, polypropylene glycol, and polybutylene glycol.

24. The prodrug of claim 21, wherein the hydrophilic moiety is a polyethylene glycol.

25. The prodrug of claim 24, wherein the polyethylene glycol comprises a monodispersed polyalkylene oxide moiety.

26. The prodrug of claim 25, wherein the monodispersed polyalkylene oxide moiety comprises from 1 to 50 alkylene oxide subunits.

27. The prodrug of claim 22, wherein R comprises a polydispersed polyalkylene oxide.

28. The prodrug of claim 27, wherein the polydispersed polyalkylene oxide is a polyethylene oxide.

29. The prodrug of claim 28, wherein the polyalkylene oxide is a straight chain, branched, graft, or fork polymer or copolymer.

30. The prodrug of claim 1, wherein R is a straight chain or branched homopolymer, random copolymer, block copolymer, graft copolymer or terpolymer selected from the group consisting of poly(oxyethylated polyols), poly(vinyl alcohol) (“PVA”), dextran, carbohydrate-based polymers, poly(oxazoline), difunctional poly(acryloylmorpholine) (“PAcM”), and poly(vinylpyrrolidone)(“PVP”).

31. The prodrug of claim 1, wherein R comprises a lipophilic moiety.

32. The prodrug of claim 31, wherein the lipophilic moiety is selected from the group consisting of C₁₋₂₀ alkyl, C₁₋₂₀ alkenyl, C₁₋₂₀ alkynyl, C₁₋₂₀ aryl, C₁₋₂₀ arylalkyl, C₁₋₂₀ alkylaryl, C₉₋₂₀ fatty acid, cholesteryl, lipophilic polymers, lipophilic oligomers and mixtures thereof.

33. The prodrug of claim 1, wherein R comprises an amphiphilic polymer or oligomer.

34. The prodrug of claim 33, wherein the amphiphilic polymer or oligomer comprises a polyethylene glycol oligomer or polymer moiety.

35. The prodrug of claim 33, wherein the amphiphilic polymer or oligomer comprises a lipophilic moiety selected from the group consisting of C₁₋₂₀ alkyl, C₁₋₂₀ alkenyl, C₁₋₂₀ alkynyl, C₁₋₂₀ aryl, C₁₋₂₀ arylalkyl, C₁₋₂₀ alkylaryl, C₉₋₂₀ fatty acid, cholesteryl, lipophilic polymers, lipophilic oligomers and mixtures thereof.

36. The prodrug of claim 1, wherein R comprises a salt-forming moiety.

37. The prodrug of claim 38, wherein the salt-forming moiety is selected from the group consisting of carboxylate and ammonium.

38. The prodrug of claim 1, wherein R is selected from the group consisting of a hydrophilic moiety, a lipophilic moiety, a salt forming moiety, and combinations thereof.

39. The prodrug of claim 1, wherein R is selected from the group consisting of (CH₂CH₂O)ₚCH₃ where p is an integer from 0 to 9; (CH₂)_{q}CH₃ where q is an integer from 1 to 9; CH₂CH₂(OCH₂CH₂)ᵣOH where r is an integer from 0 to 9; C(CH₃)₃; CH(CH₃)₂; C(CH₂OH)₃; CH(CH₂OH)₂; (CH₂CH₂O)_{y}C(O)(CH₂)_{z}CH₃ where y is an integer from 0 to 9 and z is an integer from 1 to 9; and CH₂CH₂(OCH₂CH₂)ₐC(O)(CH₂)_{b}CH₃ where a is an integer from 0 to 9 and b is an integer from 1 to 9.

40. The prodrug of claim 1, wherein R comprises a targeting moiety.

41. The prodrug of claim 40, wherein the targeting moiety is selected from the group consisting of ligands that bind to cell-surface receptors, folic acid or other epitopes, chemomimetic analogs, antibody fragments, antibodies, and arginine-glycine-aspartic acid (RGD).

42. The prodrug of claim 1, wherein the prodrug is more stable than the biologically active agent, D, in the presence of plasma, the presence of proteases, the presence of liver homogenate, the presence of acidic conditions, or the presence of basic conditions.

43. The prodrug of claim 42, wherein the prodrug is more stable than the biologically active agent, D, in the presence of plasma.

44. The prodrug of claim 1, wherein D consists essentially of 1,4-Dihydro-[3-[[[[3-[4-(3-methoxyphenyl)-1-piperidinyl]propyl]amino]carbonyl]amino]phenyl]-2,6-dimethyl-3,5-pyridinedicarboxylic acid, dimethyl esterl,4-Dihydro-[3-[[[[3-[4-(3-methoxyphenyl)-1-piperidinyl]propyl]amino]carbonyl]amino]phenyl]-2,6-dimethyl-3,5-pyridinedicarboxylic acid, dimethyl ester.

45. A pharmaceutical composition comprising the prodrug of claim 1 in combination with a pharmaceutically acceptable carrier, diluent or excipient.

46. A process for synthesizing the prodrug of claim 1, comprising:
contacting a biologically active agent, D, that comprises or is modified to comprise a functional group selected from the group consisting of amide, thioamide, imide, thioimide, urea, thiourea, carbamate, thiocarbamate, sulfonamide, and sulfonamide groups, with a compound comprising a moiety, R, wherein either D or the compound comprising R is activated such that D reacts with the compound comprising R to form a hydrolyzable carbamate bond between an NH group on the amide, thioamide, imide, thioimide, urea, thiourea, carbamate, thiocarbamate, sulfonamide, or sulfonamide groups and the compound comprising R to provide a prodrug having a hydrolyzable carbamate moiety.

47. A method of treating a subject in need of treatment, said method comprising administering an effective amount of the prodrug of claim 1.

48. The method of claim 54, wherein the prodrug is orally administered.

49. The method of claim 54, wherein the prodrug is orally administered and a pharmaceutically significant portion of the prodrug survives in the GI tract and enters the bloodstream.

50. The method of claim 54, wherein the prodrug is more stable than the biologically active agent from which it is derived in the presence of plasma, proteases, liver homogenate, acidic conditions, or basic conditions.

51. A prodrug having the following formula: 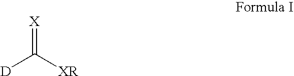
wherein:
X is O, S, or NR′;
R′is, individually, hydrogen, alkyl, substituted alkyl, aryl, substituted aryl, arylalkyl, substituted arylalkyl, alkylaryl, or substituted alkylaryl, wherein “substituted,” as applied to alkyl, aryl, alkylaryl, and arylalkyl, refers to substituents selected from alkyl, alkenyl, heterocyclyl, cycloalkyl, aryl, heteroaryl, alkylaryl, arylalkyl, halo, alkoxy, amine, trifluoroalkyl, —CN, —NO₂, —SR′, —N₃, —C(═O)NR′₂, —NR′C(═O)R″, —C(═O)R′, —C(═O)OR′, —OC(═O)R′, —NR′SO₂R′, OC(═O)NR′₂, —NR′C(═O)OR′, —SO₂R′, and —SO₂NR′₂;
D is a biologically active agent comprising a functional group selected from the group consisting of urea, thiourea, amide, thioamide, imide, thioimide, carbamate, thiocarbamate, sulfonamide, and sulfonimide, phosphoramide, or which originally included a hydroxyl, amine, thiol, and/or carboxylic acid group, where such hydroxyl, thiol or carboxylic acid group has been modified to be in the form of a urea, thiourea, amide, thioamide, imide, thioimide, carbamate, thiocarbamate, sulfonamide, or sulfonamide group;
the linkage between D and the C═X moiety is through an —N— linkage, formed from an NH group present in the urea, thiourea, amide, thioamide, imide, thioimide, carbamate, thiocarbamate, sulfonamide, or sulfonamide group in D and the C═X moiety; and
R is a modifying moiety that provides the prodrug with one or more improved pharmaceutical characteristics selected from the group consisting of improved ability of the prodrug to pass through the GI tract and enter the blood stream; improved hydrophilicity, hydrophobicity, or amphiphilicity of the prodrug; improved solubility of the prodrug in aqueous environments or organic solvents; improved ability of the prodrug to cross cell membranes; improved ability of the prodrug to cross the blood-brain barrier; improved ability of the prodrug to target a certain receptor, cell, tissue, or organ; and improved pharmacokinetic profile of the prodrug.
